(19) United States
(12) Patent Application Publication
(10) Pub. No.: US 2025/0185940 A1
(43) Pub. Date: Jun. 12, 2025

(54) HEALTH ASSESSMENT GENERATION BASED ON VOC DETECTION

(71) Applicant: Google LLC, Mountain View, CA (US)

(72) Inventor: Andrew Goldenson, Belmont, CA (US)

(73) Assignee: Google LLC, Mountain View, CA (US)

(21) Appl. No.: 18/686,361

(22) PCT Filed: Aug. 27, 2021
(86) PCT No.: PCT/US2021/048065

§ 371 Date: Feb. 23, 2024

(51) Int. Cl. A61B 5/083; A61B 5/00; A61B 5/0205; A61B 5/11; G16H 10/60; G16H 50/30

(52) U.S. Cl. CPC A61B 5/0836; A61B 5/0022; A61B 5/0205; A61B 5/4809; A61B 5/4815; A61B 5/6889; A61B 5/7275; G16H 10/60; G16H 50/30; A61B 5/1113; A61B 2505/07

(57) ABSTRACT

Techniques for creating health assessments based on Volatile Organic Compound (VOC) detection are described. In an example, a VOC sensor measures a concentration of a VOC within an enclosed space during a time period. An accumulation of carbon dioxide is detected within the space during the time period. Based on the accumulation of carbon dioxide it is determined that a human is present within the space and that the space is substantially sealed. The VOC sensor then detects that the concentration of the VOC within the space increased during the time period. A health assessment for the human is generated based on the detected increase in the VOC and a notification including the assessment is issued to an electronic device.

### CROSS-REFERENCES TO RELATED APPLICATIONS

This application is related to U.S. Application for Attorney Docket No. 094021-1252809, filed on the same day, entitled “COLLABORATIVE ENVIRONMENTAL SENSOR NETWORKS FOR INDOOR AIR QUALITY,” the disclosure of which is hereby incorporated by reference in its entirety for all purposes.

### BACKGROUND

Air quality sensors can be used to detect and monitor the concentrations of various pollutants such as particulate matter and gases. Humans can benefit from knowing the concentrations of pollutants both nearby and outside. A network of air quality sensors may be used to monitor a variety of pollutants indoors and over larger geographic regions. Monitoring one or more sensors within a network of sensors may help humans make informed decisions regarding their health and surroundings.

### SUMMARY

Various embodiments are described related to generating a health assessment based on Volatile Organic Compound (VOC) detection. In some embodiments, a method for creating health assessments from VOC detection is described. The method may comprise measuring, with a VOC sensor, a concentration of a first VOC within an enclosed space during a first time period. The method may comprise detecting an accumulation of carbon dioxide within the enclosed space during the first time period. The method may comprise determining, based on the accumulation of carbon dioxide, that a human is present within the enclosed space. The method may comprise determining, based on the accumulation of carbon dioxide, that the enclosed space is substantially sealed. When the enclosed space is substantially sealed, airflow into and out of the enclosed space may be below a threshold value. The method may comprise detecting, by the VOC sensor, that the concentration of the first VOC within the enclosed space increased during the first time period. The method may comprise generating a health assessment for the human based on the detected increase in the concentration of the first VOC. The method may comprise issuing a notification to an electronic device, the notification including the health assessment.

Embodiments of such a method may further comprise determining, based on the determination that the enclosed space is substantially sealed and the determination that the human is present within the enclosed space, that the concentration of the first VOC increased due at least in part to one or more bodily emissions by the human including exhaling, sweating, or both. The method may further comprise determining, using a sleep sensor, that the human is asleep during the first time period. The method may further comprise generating, based on sensor data collected by the sleep sensor, a sleep quality assessment for the human during the first time period. In some embodiments, generating the health assessment may be further based on a combination of the detected increase in the concentration of the first VOC and the sleep quality assessment.

In some embodiments, generating the health assessment based on the detected increase in the concentration of the first VOC may comprise identifying an increased emission of the first VOC by humans as a symptom associated with a health risk and including an identification of the health risk in the health assessment. In some embodiments, measuring the concentration of the first VOC may occur in response to detecting the accumulation of carbon dioxide within the enclosed space. In some embodiments, determining that the human is present within the enclosed space is may be further based on sensing a movement by the human using a motion sensor. In some embodiments, determining that the human is present within the enclosed space may further comprise detecting a breathing rate, heart rate, or both associated with the human.

In some embodiments, the method further comprises measuring, using an air pressure sensor, a change in air pressure within the enclosed space during the first time period. Determining that the enclosed space is substantially sealed may further comprise determining that the change in air pressure is less than a threshold value. In some embodiments, the method further comprises measuring, with the VOC sensor, concentrations of a plurality of VOCs including the first VOC.

In some embodiments, a system for creating health assessments from VOC detection is described. The system may comprise a VOC sensor configured to collect VOC concentration measurements of a first VOC within an enclosed space. The system may comprise a cloud-based health server system. The cloud-based health server system may comprise one or more processors. The cloud-based health server system may comprise a memory communicatively coupled with and readable by the one or more processors and having stored therein processor-readable instructions which, when executed by the one or more processors, cause the one or more processors to receive the VOC concentration measurements collected by the VOC sensor during a first time period. The one or more processors may determine, based on an accumulation of carbon dioxide within the enclosed space during the first time period, that a human is present within the enclosed space. The one or more processors may determine, based on the accumulation of carbon dioxide, that the enclosed space is substantially sealed. When the enclosed space is substantially sealed, airflow into and out of the enclosed space may be below a threshold value. The one or more processors may detect, from the VOC measurements, that the concentration of the first VOC within the enclosed space increased during the first time period. The one or more processors may generate, based on the detected increase in the concentration of the first VOC, a health assessment for the human. The one or more processors may issue a notification to an electronic device including the health assessment.

Embodiments of such a system may further comprise a carbon dioxide sensor configured to measure a carbon dioxide concentration within the enclosed space and transmit an indication of the accumulation of carbon dioxide to the cloud-based health server system. The system may further comprise a sleep sensor configured to determine that the human is asleep during the first time period. The system may further comprise a motion sensor configured to sense a movement by the human within the enclosed space. The system may further comprise an air pressure sensor configured to measure a change in air pressure within the enclosed space during the first time period. The system may further comprise a wearable sensor configured to detect a breathing rate, heart rate, or both associated with the human.

In some embodiments, the system may further comprise a hub device configured to receive the VOC concentration measurements from the VOC sensor and transmit the VOC concentration measurements to the cloud-based health server system. The hub device may be further configured to receive carbon dioxide measurements from a carbon dioxide sensor during the first time period and transmit an indication of the accumulation of carbon dioxide to the cloud-based health server system.

In some embodiments, a non-transitory processor-readable medium is described. The medium may comprise processor-readable instructions configured to cause one or more processors to measure a concentration of a first Volatile Organic Compound (VOC) within an enclosed space during a first time period. The one or more processors may detect an accumulation of carbon dioxide within the enclosed space during the first time period. The one or more processors may determine, based on the accumulation of carbon dioxide, that a human is present within the enclosed space. The one or more processors may determine, based on the accumulation of carbon dioxide, that the enclosed space is substantially sealed. When the enclosed space is substantially sealed, airflow into and out of the enclosed space may be below a threshold value. The one or more processors may detect that the concentration of the first VOC within the enclosed space increased during the first time period. The one or more processors may generate a health assessment for the human based on the detected increase in the concentration of the first VOC. The one or more processors may issue a notification to an electronic device, the notification including the health assessment.

In some embodiments, the one or more processors may be further configured to determine, based on the determination that the enclosed space is substantially sealed and the determination that the human is present within the enclosed space, that the concentration of the first VOC increased due at least in part to one or more bodily emissions by the human including exhaling, sweating, or both. In some embodiments, the processor-readable instructions to generate the health assessment are further configured to cause the one or more processors to identify an increased emission of the first

VOC by humans as a symptom associated with a health risk and include an identification of the health risk in the health assessment.

### BRIEF DESCRIPTION OF THE DRAWINGS

A further understanding of the nature and advantages of various embodiments may be realized by reference to the following figures. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates an embodiment of an environmental sensing system.

FIG. 2 illustrates an example of a smart home environment within which one or more of the devices, methods, systems, services, and/or computer program products described further herein can be applicable.

FIG. 3 illustrates an embodiment of an air quality system for managing a distributed environmental sensor network.

FIG. 4 illustrates an embodiment of an air quality sensor system in a distributed environmental sensor network.

FIG. 5 illustrates an example environment within which a distributed environmental sensor network may be deployed to detect endogenous air pollution within a structure.

FIG. 6 illustrates another example environment within which a distributed environmental sensor network may be deployed to detect exogenous air pollution within a structure.

FIG. 7 illustrates a graph of historical air quality.

FIG. 8 illustrates an embodiment of an interface for monitoring a distributed environmental sensing network.

FIG. 9 illustrates an embodiment of a method for managing a distributed environmental sensor network.

FIG. 10 illustrates an embodiment of a system for generating health assessments based on detected volatile organic compounds.

FIG. 11 illustrates an example of an environment within which one or more of the devices, methods, systems, services, and/or computer program products described further herein can be applicable.

FIG. 12 illustrates a graph of carbon dioxide and VOC concentrations detected in an enclosed space.

FIG. 13 illustrates an embodiment of an interface for viewing generated health assessments based on detected volatile organic compounds.

FIGS. 14A and 14B illustrate an embodiment of a method for generating a health assessment based on detected volatile organic compounds.

### DETAILED DESCRIPTION

As the number of connected devices in the Internet of things (IoT) increases, managing the ever increasing generation of data in a meaningful and useful way for society can often be a challenging feat. Nearly every aspect of our daily life and environment can be monitored in some way, creating access to new data like never before. Monitoring air quality is no exception. Pollution and poor air quality could be from any number of sources such as a forest fire, a backyard barbecue, changing weather conditions, a gas leak in a home, and industrial pollution, among many other sources. An air quality sensor can monitor and detect a host of pollutants such as carbon dioxide, carbon monoxide, lead, chemicals, and organic compounds, among many others within a close proximity to the sensor. The data collected by multiple air quality sensors could be used in order to make informed decisions or preemptive actions regarding the quality of air a person is breathing or about to breathe.

After receiving explicit permission to gather and/or share air quality data collected by a network of air quality sensors inside and outside homes, air quality data covering a broader geographic area can be shared to inform people before a pollutant reaches them and may allow them to take preemptive actions such as closing windows or turning off external ventilation systems. Similarly, air quality data covering a broader geographic area can be shared, after receiving explicit permission from each user, to inform people that outside air is cleaner or healthier than the air quality inside a building, allowing them to take remediation action such as opening windows or turning on external ventilation systems and/or air purification systems. Over a sufficient amount of time, the collected air quality data from the network of air quality sensors may be used to generate predictions about future air quality. By identifying trends in detected pollutants or lower air quality at certain times of the day, preemptive actions may be taken such as avoiding the use of external ventilation systems during those times of day.

In addition, a network of air quality sensors may be used to identify or otherwise locate the source of a pollutant. Using the detection times and relative distances between disparate sensors, a location of the source of a pollutant may be estimated. Similarly, as more sensors detect the pollutant in varying levels moving away from the source, a prediction regarding the potentially affected area may be generated. These determinations may be supplemented with commercial or governmental weather and air quality data.

Following a user granting permission for air quality data from a sensor to be used anonymously, the air quality data generated by the air quality sensor may be tagged with a rough geographic location such as a zip code, a city, a neighborhood, or a perimeter. By tagging the data with a rough geographic location as opposed to a specific address or location, the air quality data can be populated to the cloud and shared without personally identifiable information (PII), thereby preserving the privacy of each individual person associated with a sensor. After collecting and analyzing the air quality data, alerts and notifications may be sent to people who may be potentially affected by poor air quality or pollutants. The alerts and notifications may be sent to an electronic device associated with a user account managed by a central server system.

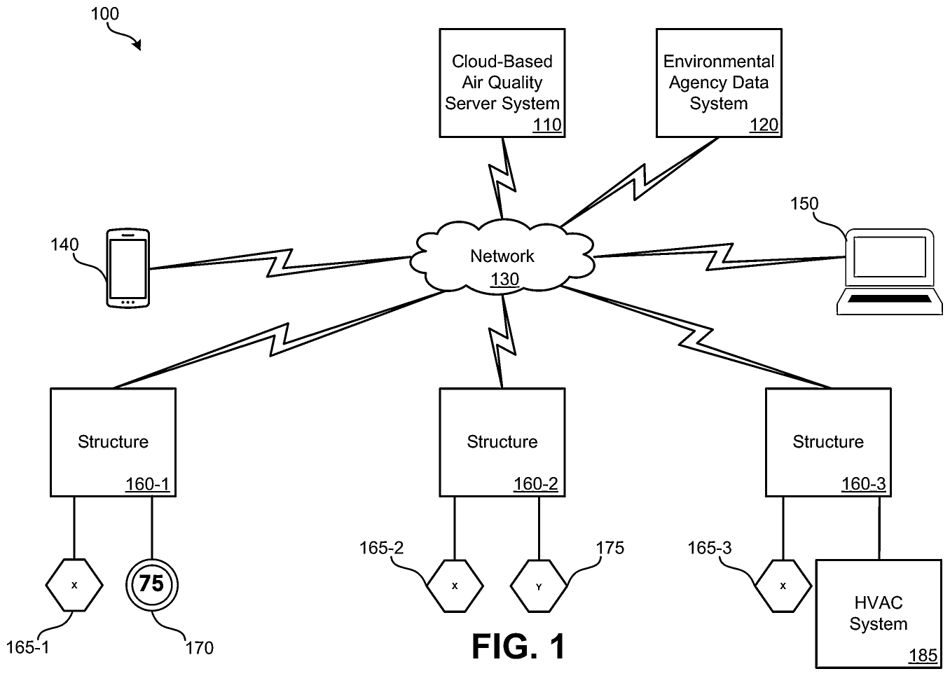

Further detail regarding the collection and management of air quality data from a network of air quality sensors is provided in relation to the figures. FIG. 1 illustrates an embodiment of an environmental sensing system 100. System 100 can include: cloud-based air quality server system 110; environmental agency data system 120; network 130; mobile device 140; personal computer 150; and structures 160. Structures 160 may include, or otherwise be associated with, one or more of: air quality sensors 165; smart thermostat 170; Volatile Organic Compound (VOC) sensor 175; and HVAC system 185. In some embodiments, one or more of the components of system 100 may be communicatively connected to other components of system 100 via network 130.

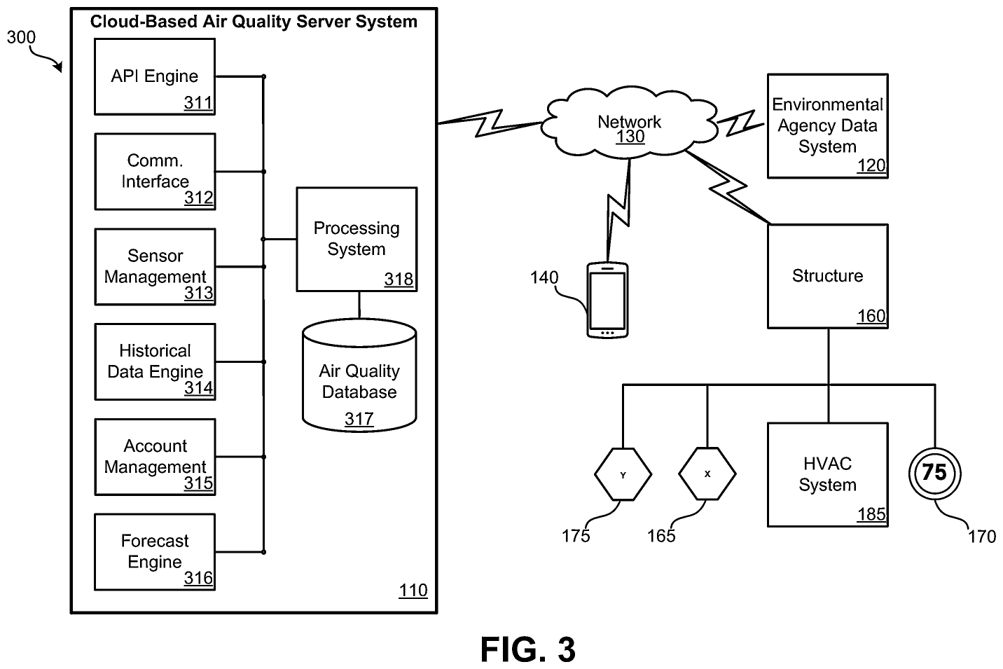

Cloud-based air quality server system 110 can include one or more processors configured to perform various functions, such as receive indications of detected air pollutants, as further described in relation to FIG. 3, infra. Cloud-based air quality server system 110 can include one or more physical servers running one or more processes. Cloud-based air quality server system 110 can also include one or more processes distributed across a cloud-based server system. In some embodiments, cloud-based air quality server system 110 is connected over network 130 to any or all of the other components of system 100. For instance, cloud-based air quality server system 110 may connect to air quality sensor 165-1 to receive an indication that a pollutant is present within structure 160-1. As another example, cloud-based air quality server system 110 may connect to air quality sensor 165-2 to cause it to change operating modes.

Cloud-based air quality server system 110 may also connect to mobile device 140 and personal computer 150 to send updates or notifications about the current air quality. For example, after receiving an indication from air quality sensor 165-1 that a pollutant is present within structure 160-1, cloud-based air quality server system 110 may send a notification to mobile device 140 with an alert indicating that the pollutant was detected within structure 160-1. Cloud-based air quality server system 110 may also connect to smart thermostat 170 to send commands indicating how and/or when to control HVAC system 185. For example, cloud-based air quality server system 110 may send a command to smart thermostat 170 instructing it to activate or deactivate an outside ventilation component of HVAC system 185, activate a fan, and/or activate heating or cooling.

Environmental agency data system 120 can be a server system, such as a cloud based server system, connected through network 130 and may be capable of generating and distributing publicly available environmental data. The environmental data may include weather data such as the temperature, wind speed and direction, humidity and the like. The environmental data may also include air quality data, such as an air quality index (AQI). The air quality data may include information about the current and predicted air quality for one or more regions. Alternatively, the air quality data may include specific information about environmental accidents or the sources of a particular pollutant within a region. For example, the air quality data may indicate that a natural gas tanker was involved in an accident on a nearby highway and the gases are dispersing throughout a nearby area. The air quality data provided by environmental agency data system 120 may be used by cloud-based air quality server system 110 to generate notifications and/or alerts to users. Cloud-based air quality server system 110 may also use the air quality data to generate and/or update predictions regarding the potential air quality for a region or area.

Environmental agency data system 120 may provide the air quality data as a web service using a published Application Programming Interface (“API”). For example, environmental agency data system 120 may publish an API allowing external systems, such as cloud-based air quality server system 110, to connect to it over network 130 in order to send requests for data and receive the requested data in response. Alternatively or in addition, Environmental agency data system 120 may publish updated air quality data for various regions to subscriber services.

Network 130 can include one or more wireless networks, wired networks, public networks, private networks, and/or mesh networks. A home wireless local area network (e.g., a Wi-Fi network) may be part of network 130. Network 130 can include the Internet. Network 130 can include a mesh network, such as Thread, which may include one or more other smart home devices, and may be used to enable air quality sensors 165, smart thermostat 170, and VOC sensor 175 to communicate with another network, such as a Wi-Fi network. Any of air quality sensors 165, smart thermostat 170, and

VOC sensor 175 may function as an edge router that translates communications received from other devices on a relatively low power mesh network to another form of network, such as a relatively higher power network, such as a Wi-Fi network.

Mobile device 140 may be a smartphone, tablet computer, laptop computer, gaming device, or some other form of computerized device that can communicate with cloud-based air quality server system 110 via network 130 or can communicate directly with any of air quality sensors 165, smart thermostat 170, and VOC sensor 175. Similarly, personal computer 150 may be a laptop computer, desktop computer, or some other computerized device that can communicate with cloud-based air quality server system 110 via network 130 or can communicate directly with any of air quality sensors 165, smart thermostat 170, and VOC sensor 175. A user can interact with an application executed on mobile device 140 or personal computer 150 to control, view data from, or interact with air quality sensors 165, smart thermostat 170, and VOC sensor 175. For example, the user of mobile device 140, or personal computer 150, can connect via network 130 to smart thermostat 170 at the user's home to monitor the status of smart thermostat 170 or send heating and cooling instructions to smart thermostat 170 that will in turn cause an HVAC system to provide heating or cooling to the user's home. As another example, mobile device 140 may connect via network 130 to air quality sensors 165 and/or VOC sensor 175 to monitor the air quality within and/or around the user's home. Mobile device 140 may also be connected over network 130 to cloud-based air quality server system 110. For example, cloud-based air quality server system 110 may send notifications to mobile device 140 about the air quality surrounding or inside the user's home or location. The notifications or updates may be in the form of a text message, an email, or a notification through an application.

Structures 160 can be one or more structures and/or buildings of various types. For example structure 160-1 may be a residential dwelling such as a house, apartment, and/or recreational vehicle (RV). As another example, structure 160-2 may be a multifamily housing structure such as an apartment or condominium building. In this example, structure 160-2 may include multiple substructures, such as an apartment unit. In yet another example, structure 160-3 may be a commercial structure such as an office building or industrial complex with one or more air quality sensors disposed therein, such as air quality sensor 165-3.

Structures 160 may be associated with one or more residential user accounts managed by cloud-based air quality server system 110. For example, a homeowner may create a residential user account associated with structure 160-1 via mobile device 140 and/or personal computer 150 on cloud-based air quality server system 110. A residential user account may include various information about structures 160, such as the size, location, number of rooms, the existence and placement of sensors, such as air quality sensors 165 and/or VOC sensors 175, and the like. In some embodiments, a structure 160 may be associated with multiple residential user accounts. For example, structure 160-2 may be an apartment building with multiple apartments each associated with a separate residential user account. Mobile device 140 and/or personal computer 150 may also be associated with a residential user account. For example, after receiving an indication of the detection of the first pollutant within and/or around a structure, cloud-based air quality server system 110 may send a notification to mobile device 140 and/or personal computer 150 associated with a residential user account that is also associated with the structure. Residential user accounts may be any type of user account and does not need to specifically be for residential purposes. For example, a user account may be created in order to access any number of services provided by a cloud-based server system. The individual user may then choose to use those services for any purpose, such as residential and/or commercial purposes.

Structures 160 can include one or more of air quality sensors 165, smart thermostat 170, VOC sensor 175, and or HVAC system 185. For example, structure 160-1 may be a house and may include one or more air quality sensors 165 and/or VOC sensors 175 disposed throughout the interior of the structure as well as around an exterior of the structure. Structure 160-1 may also include smart thermostat 170 coupled to HVAC system 185. As another example, structure 160-1 may be an apartment building and may include one or more air quality sensors 165 and/or VOC sensors 175 in each unit, in the interior common areas, and exterior locations, such as a parking garage, pool area, and/or playground area.

Air quality sensors 165 may be any device capable of measuring air pollution and connecting to network 130. Air quality sensors 165 may include one or more processors that may execute special-purpose software stored in a memory of the device. Air quality sensors 165 may measure one or more types of pollution, such as but not limited to, gases, chemicals, organic compounds, and/or particulate matter. For example, air quality sensors 165 may include one or more individual sensors calibrated to detect a specific pollutant. Each air quality sensor 165 may measure one or more pollutants at the same time and/or be specific to one type of particular pollutant. For example, VOC sensor 175 may be an air quality sensor designed to detect and monitor VOC concentrations only. In some embodiments, air quality sensors 165 may only detect the presence of a pollutant. For example, air quality sensors 165 may have threshold values for each detectable pollutant and may only indicate that a pollutant was detected if the concentration of the pollutant is above the threshold value. In some embodiments air quality sensors 165 may measure the concentration of pollutants. For example, each air quality sensor 165 and/or VOC sensor 175 may be capable of measuring the parts per million (PPM) and/or parts per billion (PPB) by volume of various air pollutants.

Air quality sensors 165 and/or VOC sensor 175 may connect via network 130 to one or more additional components of system 100. In some embodiments, air quality sensors 165 and/or VOC sensor 175 may connect via network 130 to cloud-based air quality server system 110. For example, air quality sensor 165-1 may transmit an indication that a first pollutant was detected to cloud-based air quality server system 110 via network 130. In some embodiments, an indication that a pollutant was detected may include one or more additional pieces of information such as, a location of detection, a time of detection, and/or an amount of pollutant detected. Air quality sensors 165 may transmit an indication as soon as a pollutant is detected and/or they may collect data over a predetermined time interval and transmit the collected data at the end of the predetermined time interval. Air quality sensors 165 and/or VOC sensor 175 may connect via network 130 to mobile device 140 and/or personal computer 150. For example, a user of mobile device 140 may connect to any one or more air quality sensors 165 to investigate the quality of air surrounding the sensors. Air quality sensors 165 and/or VOC sensor 175 and additionally connect to other air quality sensors 165 and/or VOC sensor 175.

Smart thermostat 170 can be a smart thermostat capable of connecting to network 130 and controlling HVAC system 185. Smart thermostat 170 may include one or more processors that may execute special-purpose software stored in a memory of smart thermostat 170. Smart thermostat 170 can include one or more sensors, such as a temperature sensor or an ambient light sensor. Smart thermostat 170 can also include an electronic display. The electronic display may include a touch sensor that allows a user to interact with the electronic screen. Smart thermostat 170 may connect via network 130 to cloud-based air quality server system 110. For example, smart thermostat 170 may receive instructions to control HVAC system 185 based on the quality of air within and/or surrounding structures 160. In some embodiments, smart thermostat 170 may connect via network 130 to mobile device 140 or personal computer 150. For example, smart thermostat 170 may receive heating or cooling instructions from a user's mobile device 140 or personal computer 150.

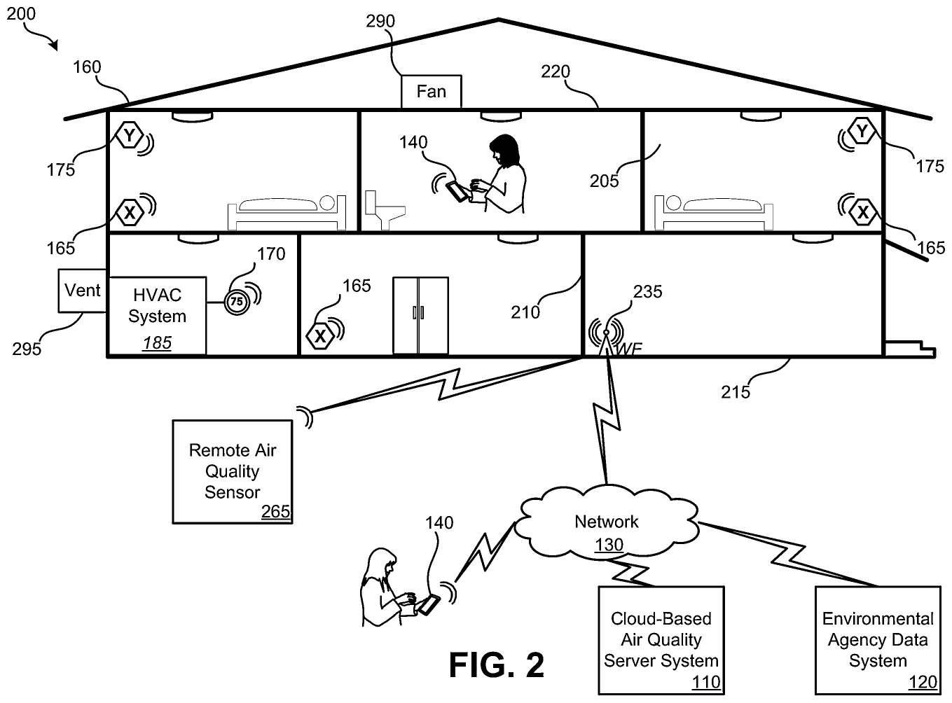

FIG. 2 illustrates an example of a smart home environment 200 within which one or more of the devices, methods, systems, services, and/or computer program products described further herein can be applicable. The depicted smart home environment 200 includes structure 160. Structure 160 can include, e.g., a house, condominium, apartment, office building, garage, or mobile home as described above. The smart home environment may include devices, such as air quality sensors 165, VOC sensors 175, smart thermostat 170, and wireless router 235 inside and/or outside of the actual structure 160. For example, one or more remote air quality sensors 265 can be located outside of structure 160.

The depicted structure 160 includes a plurality of rooms 205, separated at least partly from each other via walls 210. Walls 210 can include interior walls or exterior walls. Each room can further include a floor 215 and a ceiling 220. Devices can be mounted on, integrated with and/or supported by a wall 210, floor 215 or ceiling 220.

The smart home depicted in FIG. 2 includes a plurality of devices, including intelligent, multi-sensing, network-connected devices that can integrate seamlessly with each other and/or with cloud-based server systems to provide any of a variety of useful smart home objectives. One, more or each of the devices illustrated in the smart home environment and/or in the figure can include one or more sensors, a user interface, a power supply, a communications component, a modularity unit and intelligent software as described herein. Examples of devices are shown in FIG. 2.

An intelligent, multi-sensing, network-connected thermostat, such as smart thermostat 170, can detect ambient climate characteristics (e.g., temperature and/or humidity) and control a heating, ventilation and air-conditioning (HVAC) system 185. HVAC system 185 may be coupled with and/or be capable of controlling fan 290 and/or vent 295. Alternatively or in addition smart thermostat 170 may be configured to control fan 290 and or vent 295. For example, either HVAC system 185 or smart thermostat 170 may be configured to activate fan 290 and/or vent 295 in order to draw external air in through the vent 295 and exhaust the internal air through fan 290. One or more intelligent, network-connected, multi-sensing devices, such as air quality sensors 165 and/or VOC sensors 175, can detect the presence of a hazardous substance and/or pollutant in and around the home environment (e.g., smoke, carbon monoxide, methane, radon, acetone, and the like).

In addition to containing processing and sensing capabilities, each of the devices, such as air quality sensors 165, VOC sensors 175, remote air quality sensor 265, and/or smart thermostat 170, can be capable of data communications and information sharing with each of the other devices, as well as to any cloud server or any other device that is network connected anywhere in the world, such as mobile device 140 and/or personal computer 150 as described above. The devices can send and receive communications via any of a variety of custom or standard wireless protocols (Wi-Fi, ZigBee, 6LoWPAN, Thread, Bluetooth, BLE, HomeKit Accessory Protocol (HAP), Weave, etc.) and/or any of a variety of custom or standard wired protocols (CAT6 Ethernet, HomePlug, etc.). Each of the devices may also be capable of receiving voice commands or other voice-based inputs from a user, such as the Google Home® interface.

For example, a first device can communicate with a second device via a wireless router 235. A device can further communicate with remote devices via a connection to a network, such as the network 130. Through the network 130, the device can communicate with a central server or a cloud-computing system, such as cloud-based air quality server system 110 and/or environmental agency data system 120. Further, software updates can be automatically sent from the central server or cloud-computing system to the devices (e.g., when available, when purchased, or at routine intervals).

By virtue of network connectivity, one or more of the smart-home devices of FIG. 2 can further allow a user to interact with the device even if the user is not proximate to the device. For example, a user can communicate with a device such as mobile device 140 and/or personal computer 150. A webpage or app can be configured to receive communications from the user and control the device based on the communications and/or to present information about the device's operation to the user. For example, the user can view a current setpoint temperature for a device and adjust it using a computer. The user can be in the structure during this remote communication or outside the structure.

FIG. 3 illustrates an embodiment of an air quality system 300 for managing a distributed environmental sensor network. Air quality system 300 can include: cloud-based air quality server system 110; environmental agency data system 120; network 130; mobile device 140; and structure 160. Structure 160 may include any one or more of: air quality sensors 165, VOC sensors 175, smart thermostat 170, and HVAC system 185. While only one structure 160 as illustrated in FIG. 3, it should be understood that air quality system 300 may include a plurality of structures similar to structure 160. Each sensing component included in each structure of a plurality of structures may form a distributed environmental sensor network controlled and/or managed by cloud-based air quality server system 110. Environmental agency data system 120 may function as detailed in relation to FIG. 1, supra. Smart thermostat 170 and HVAC system 185 may function as detailed in relation to FIG. 1, supra. Network 130 may function as detailed in relation to FIG. 1, supra.

Cloud-based air quality server system 110 can include a plurality of services such as: API engine 311; communication interface 312; sensor management module 313; historical data engine 314; account management module 315; and forecast engine 316. Cloud-based air quality server system 110 can also include one or more databases such as air quality database 317. Cloud-based air quality server system 110 can also include processing system 318 that can coordinate the execution of the various functionalities provided by the plurality of services and can communicate with the one or more databases such as air quality database 317.

API engine 311 may implement published interfaces from one or more external systems and devices. The published interfaces may allow cloud-based air quality server system 110 to interact with various external systems to request and exchange data, such as environmental agency data system 120. API engine 311 may also allow cloud-based air quality server system 110 to communicate with various devices connected to network 130. For example, API engine 311 may implement an interface for sending text messages, emails, or application notifications to mobile device 140. API engine 311 may also configure cloud-based air quality server system 110 to send requests for air quality indications from one or more air quality sensors, such as air quality sensor 165 and/or VOC sensor 175. API engine 311 may also allow cloud-based air quality server system 110 to send instructions for operating smart devices connected to network 130. For example, API engine 311 may implement an interface for smart thermostat 170.

Communication interface 312 may be used to communicate with one or more wired networks. In some embodiments, a wired network interface may be present, such as to allow communication with a local area network (LAN). Communication interface 312 may also be used to communicate with distributed services across multiple virtual machines through a virtual network. Communication interface 312 may be used by one or more of the other processes in order to communicate with the other process or with external devices and services such as mobile device 140, environmental agency data system 120, air quality sensor 165, VOC sensor 175, or smart thermostat 170.

Sensor management module 313 may include one or more processes for managing a distributed environmental sensing network. For example, sensor management module 313 may request and receive status updates from each of a plurality of environmental sensors. The environmental sensors may include air quality sensor 165, VOC sensor 175, smart thermostat 170, air pressure sensors, carbon dioxide sensors, ambient light sensors, motion detection sensors, and the like. The status updates received from the plurality of environmental sensors may include data collected by the plurality of environmental sensors. For example, a status update may include an indication that a pollutant was detected within a first structure and/or the vicinity of the first structure. As another example, a status update may include the concentration of the pollutant within the first structure.

Status updates may also include settings associated with the particular sensor that transmitted the update. For example, a status update may include such settings as the location of the sensor and or the time at which the sensor data was collected. In some embodiments, the location of the sensor is determined based on the identification of the sensor. For example, after receiving a status update from the sensor, sensor management module 313 may determine the approximate location of the sensor by looking up the sensor ID in a table or database mapping sensor IDs to residential user accounts and/or approximate geographic locations.

Sensor management module 313 may also analyze a collection of status updates to identify potential follow-up actions. Follow-up actions may include generating notifications, controlling individual environmental sensors, sending instructions to smart devices, and the like. For example, sensor management module 313 may determine the existence of low air quality and/or a particular pollutant within a geographic region and generate notifications to residential user accounts with structures and/or mobile devices associated with the geographic region. As another example, after determining the existence of low air quality and/or particular pollutant within a geographic region, sensor management module 313 may transmit instructions to smart devices, such as smart thermostat 170, to deactivate an external air ventilation component of HVAC systems within structures located in the vicinity of the geographic region.

Sensor management module 313 may also analyze the collection of status updates, including indications of the detection of one or more pollutants, to determine a potential source of the one or more pollutants. For example, sensor management module 313 may receive an indication that a first pollutant was detected within a first structure. Sensor management module 313 may also receive an indication that the first pollutant was not detected within a second structure in the vicinity of the first structure. Alternatively, sensor management module 313 may determine from the absence of an indication that the first pollutant was detected within the second structure that the first pollutant is not present within the second structure. Based on the determination that the first pollutant is within the first structure, but not the second structure, sensor management module 313 may determine that the source of the first pollutant is within the first structure. After determining that the source of the first pollutant is likely within the first structure, sensor management module 313 may generate and transmit a notification to a residential user account associated with the first structure indicating a potential endogenous source of the first pollutant within the first structure.

As another example, after determining that the first pollutant is within the first structure, sensor management module 313 may determine that the first pollutant is also within the second structure. Based on the determination that the first pollutant is within both the first structure and the second structure, sensor management module 313 may determine that the source of the first pollutant is outside both the first structure and the second structure. After determining that the source of the first pollutant is likely outside both the first structure and second structure, sensor management module 313 may generate and transmit a notification to one or more residential user accounts associated with the first structure, the second structure, and/or additional structures within the vicinity of the first structure and the second structure.

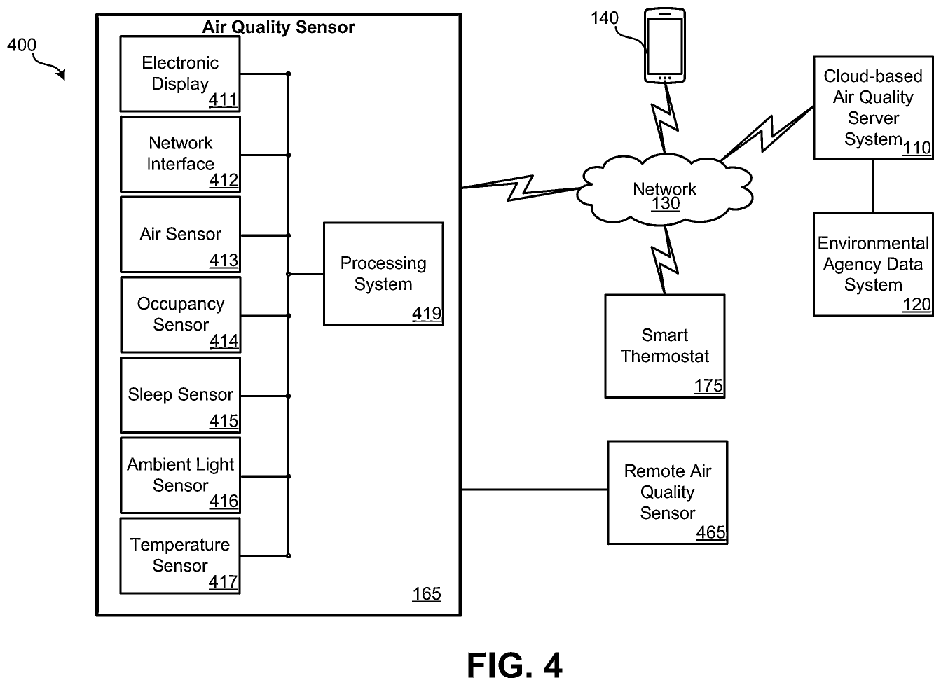

Sensor management module 313 may also control the operation of individual environmental sensors in the distributed environmental sensing network. In some embodiments, sensor management module 313 may be configured to change an operational mode of air quality sensor 165 from a first operational mode to another of multiple potential operational modes, as discussed further below in relation to FIG. 4, infra. For example, after receiving an indication from an air quality sensor located within a first structure, sensor management module 313 may cause an air quality sensor located within a second structure near the vicinity of the first structure to change operational modes from a normal sensitivity mode to a high-sensitivity mode. As another example, sensor management module 313 may be configured to send instructions to smart thermostat 170 to activate and/or deactivate an external air ventilation component of HVAC system 185.

Historical data engine 314 may include processes for analyzing historical data and metrics. In some embodiments, historical data engine 314 periodically or occasionally analyzes historical air quality data within various regions and/or structures to help predict when air quality will rise or fall again in the future. For example, historical data engine 314 may analyze historical air quality data for structures within the vicinity of the highway and determine that the concentration of one or more pollutants increases and decreases during predictable time intervals each day coinciding with rush-hour traffic. As another example, historical data engine 314 may analyze historical air quality data for a set of sensors within a single structure and determine that there is a predictable rise and fall of carbon dioxide within the structure in the evenings coinciding with when inhabitants of the structure are present. The trends and predictions identified by historical data engine 314 may be used to generate notifications to residential user account associated with the geographic region and/or structure. Alternatively, or in addition, the trends and predictions identified by historical data engine 314 may be provided to forecast engine 316 for further analysis and notification generation. Notifications may include a summary of the historical data and/or suggestions for adjusting daily activities, such as when to open and/or close windows in a house.

Account management module 315 may include one or more processes for managing residential user accounts. For example, account management module 315 may access, modify, and store account details for a specific residential user account such as information for one or more devices owned and operated by one or more users associated with the account, the rough geographic location of the devices and structures associated with a residential user account, and the like. Account management module 315 may provide residential user account-specific information to either or both of sensor management module 313, historical data engine 314, and forecast engine 316 to generate user account-specific notifications. In some embodiments, account management module 315 may also send communications to a user associated with a user account, such as notifications or updates, or to an application on a mobile device 140 associated with the user account. For example, account management module 315 may send an email, text, or application notification to a residential user account indicating the air quality within or around a structure associated with the residential user account.

Forecast engine 316 may include one or more processes for analyzing air quality data and generating air quality forecasts. Forecast engine 316 may receive current air quality data from sensor management module 313 and/or historical air quality data from historical data engine 314. Forecast engine 316 may also receive current and/or historical air quality data from environmental agency data system 120. The current and/or historical air quality data may include the raw data collected by each individual sensor of the distributed environmental sensor network. Alternatively the current and/or historical air quality data may include a summary of the raw data collected by each individual sensor distributed environmental sensor network. For example, historical data engine 314 may analyze indications that pollutants were detected, and/or the concentrations of the pollutants, and generate a summary of the data to be provided to forecast engine 316. In some embodiments, forecast engine 316 generates multiple forecasts for a single region in addition to multiple forecasts from multiple regions. For example, forecast engine 316 may generate an air quality forecast for a city or municipality as well as multiple forecasts for individual structures within the city or municipality.

Forecast engine 316 may generate forecasts using either the air quality data collected from the distributed environmental sensor network, environmental agency data system 120, or both. For example, forecast engine 316 may generate an initial forecast using only data collected from the distributed environmental sensor network and supplement the generated forecast with data collected from environmental agency data system 120 as it becomes available.

One or more databases, such as air quality database 317, may store or otherwise make data accessible to cloud-based air quality server system 110. Air quality database 317 may include data associated with historical and predicted air quality. The historical air quality data may include both the air quality collected by the distributed environmental sensor network or third-party services for a city or region, such as data collected from environmental agency data system 120. The one or more databases, including air quality database 317, may be implemented by one or more suitable database structures such as a relational database (e.g., SQL) or a NoSQL database (e.g., MongoDB).

Processing system 318 can include one or more processors. Processing system 318 may include one or more special-purpose or general-purpose processors. Such special-purpose processors may include processors that are specifically designed to perform the functions detailed herein. Such special-purpose processors may be ASICs or FPGAs which are general-purpose components that are physically and electrically configured to perform the functions detailed herein. Such general-purpose processors may execute special-purpose software that is stored using one or more non-transitory processor-readable mediums, such as random access memory (RAM), flash memory, a hard disk drive (HDD), or a solid state drive (SSD) of cloud-based air quality server system 110.

FIG. 4 illustrates an embodiment of an air quality sensor system 400 in a distributed environmental sensor network. Air quality sensor system 400 can include air quality sensor 165, smart thermostat 170; network 130; cloud-based air quality server system 110; mobile device 140; and remote air quality sensor 465. Cloud-based air quality server system 110 may function as described in relation to FIGS. 1-3, supra. Network 130 may function as described in relation to FIG. 1, supra. Environmental agency data system 120 may be connected to cloud-based air quality server system 110 and may function as described in relation to FIG. 1, supra. Smart thermostat 170 may function as described in relation to FIGS. 1-3, supra. Air quality sensor system 400 can include a plurality of air quality sensors 165. The plurality of air quality sensors 165 may form the distributed environmental sensing network.

Air quality sensor 165 can include multiple components, such as: electronic display 411; network interface 412; air sensor 413; occupancy sensor 414; sleep sensor 415; ambient light sensor 416; temperature sensor 417; and processing system 419. In some embodiments, air quality sensor 165 includes a subset of components in a single device while other components are housed in distributed devices. For example, air quality sensor 165 may include electronic display 411, network interface 412, air sensor 413, and processing system 419, while the remaining components, such as occupancy sensor 414, sleep sensor 415, ambient light sensor 416, and temperature sensor 417 may be housed in one or more distinct devices. In this example, the one or more distinct devices may include individual displays, network interfaces, and processing systems in order to communicate with air quality sensor 165 and others of the one or more distinct devices. Air quality sensor 165 may also connect to one or more remote air quality sensors, such as remote air quality sensor 465. In some embodiments remote air quality sensor 465 may include one or more of the same features of air quality sensor 165 and/or function in a similar manner as air quality sensor 165.

Air quality sensor 165 may include multiple operating modes. For example, the operating modes may include a low power mode, a normal-sensitivity mode, and a high-sensitivity mode. When operating in each mode, air quality sensor 165 may modify and/or adjust the sampling rate of one or more of the sensing components. For example, in a low-power mode, the sampling rate may be in the range of once every five minutes to as low as once every hour or longer in order to reduce power consumption by air quality sensor 165. As another example, in a normal-sensitivity mode, the sampling rate may be in the range of once every thirty minutes to as high as once every five minutes or less in order to balance power consumption with accurate sensor measurements. In yet another example, the sampling rate for a high-sensitivity mode may be in the range of once every five minutes to as high as 10 Hz in order to maximize the accuracy of sensor measurements taken by air quality sensor 165.

The operating mode of air quality sensor 165, and/or the sampling rate, may change based on the detection of pollutants. For example, after detecting the presence of a pollutant, air quality sensor 165 may change from a first operating mode, such as the normal-sensitivity mode, to a second operating mode, such as the high-sensitivity mode. Changing from the first operating mode to the second operating mode may enable the system to more accurately monitor the levels of the pollutant over time and/or provide more real time updates as to whether the pollutant is still present within the environment, or if it has dissipated. Similarly, the operating mode may change in response to remediation activity. For example, after detecting a pollutant within a structure and causing an external ventilation system and/or air purifier system to activate, the operating mode may change to the high-sensitivity mode in order to monitor the rate at which the pollutant dissipates from the environment and/or when the pollutant is no longer present.

In some embodiments, the operating modes may alter or adjust various threshold values. For example, in a normal-sensitivity mode, air sensor 413 may indicate the presence of a pollutant when the concentration of the pollutant rises above a first threshold value. In a high-sensitivity mode, air sensor 413 may indicate the presence of the pollutant when the concentration of the pollutant rises above a second threshold value. The second threshold value may be lower than the first threshold value in order to detect the pollutant before it reaches the first threshold concentration value.

In some embodiments, each component of air quality sensor 165, such as air sensor 413, occupancy sensor 414, sleep sensor 415, ambient light sensor 416, and temperature sensor 417, may have a distinct operating mode. For example, air sensor 413 may be configured to operate in a normal-sensitivity mode while other components, such as sleep sensor 415, are configured to operate in a low-power mode.

Electronic display 411 may be a display such as a liquid crystal display, a light emitting diode display, or any other similar display configured to display information produced by air quality sensor 165. In some embodiments, electronic display 411 is only visible when electronic display 411 is illuminated. In some embodiments, electronic display 411 is a touch screen. A touch sensor may allow one or more gestures, including tap and swipe gestures, to be detected. Electronic display 411 may display one or more pieces of information generated by air quality sensor 165. For example, electronic display 411 may display status of air quality sensor 165, one or more air quality measurements, such as the concentrations of one or more pollutants, and the like.

Network interface 412 may be used to communicate with one or more wired or wireless networks. Network interface 412 may communicate with a wireless local area network such as a Wi-Fi network. Additional or alternative network interfaces may also be present. For example, air quality sensor 165 may be able to communicate with a user device directly, such as by using Bluetooth®. Air quality sensor 165 may be able to communicate via a mesh network with various other home automation devices. Mesh networks may use relatively less power compared to wireless local area network-based communication, such as Wi-Fi. In some embodiments, air quality sensor 165 can serve as an edge router that translates communications between a mesh network and a wireless network, such as a Wi-Fi network. In some embodiments, a wired network interface may be present, such as to allow communication with a local area network (LAN). One or more direct wireless communication interfaces may also be present, such as to enable direct communication with a remote air quality sensor, such as remote air quality sensor 465, installed in a different location distinct from air quality sensor 165. The evolution of wireless communication to fifth generation (5G) and sixth generation (6G) standards and technologies provides greater throughput with lower latency which enhances mobile broadband services. 5G and 6G technologies also provide new classes of services, over control and data channels, for vehicular networking (V2X), fixed wireless broadband, and the Internet of Things (IoT). Air quality sensor 165 may include one or more wireless interfaces that can communicate using 5G and/or 6G networks.

Air sensor 413 may be one or more sensors configured to detect the presence of various airborne pollutants and/or measure the concentration of such pollutants. Examples of pollutants air sensor 413 may be able to detect include gases (e.g., ammonia, carbon monoxide, sulfur dioxide, methane, carbon dioxide, etc.), particulates (e.g., aerosols), and/or biological molecules. Air sensor 413 may indicate when the concentration of one or more types of pollutants rise above a certain threshold concentration. Alternatively or in addition, air sensor 413 may be configured to measure the actual concentration of various types of pollutants. The concentration of pollutants may be measured in parts per million, parts per billion, or any similar unit of measure for the concentration of airborne pollutants. In some embodiments, air sensor 413 may be configured to generate an overall air quality score based on the concentrations of one or more air pollutants measured by air sensor 413. For example, air sensor 413 may score the surrounding air using an air quality index (AQI), or any similar measure of air quality.

Occupancy sensor 414 may be one or more sensors configured to detect the presence of one or more humans within the vicinity of occupancy sensor 414. For example, occupancy sensor 414 may include one or more of a radar sensor, a lidar sensor, a photographic sensor, an infrared sensor, or any other similar sensor capable of detecting motion within an environment. Alternatively or in addition, occupancy sensor 414 may include a carbon dioxide sensor. For example, by detecting the concentration of carbon dioxide within an environment, occupancy sensor 414 may be able to determine that one or more humans are within the environment due to an observed increase in the concentration of carbon dioxide within the environment.

Sleep sensor 415 may be one or more sensors configured to detect when a person is sleeping and monitor the quality of sleep. For example, sleep sensor 415 may include one or more of a heart rate monitor, a breathing rate monitor, a brain activity monitor, a motion detection sensor, and eye activity monitor or any other similar sensor capable of monitoring and detecting measurable characteristics of the sleeping human. In some embodiments, measurements from sleep sensor 415 may be used to alter a response to the detection of a pollutant by air sensor 413. For example, after detecting the presence of the first pollutant by air sensor 413 air quality sensor 165 may determine from inputs generated by sleep sensor 415 that an occupant is asleep and to determine whether to generate an alarm based on the severity of the detected pollutant.

Ambient light sensor 416 may sense the amount of light present in the environment of air quality sensor 165. Measurements made by ambient light sensor 416 may be used to adjust the brightness of electronic display 411. Measurements made by ambient light sensor 416 may be used by occupancy sensor 414 and/or sleep sensor 415 to determine whether humans are present and/or when a human may be asleep. For example, ambient light sensor 416 may detect light present in the environment of air quality sensor 165 during the time of day when natural light would otherwise not be present, thereby indicating that a human is present and has turned lights on. As another example, ambient light sensor 416 may detect the presence of light in a room when a human would otherwise be sleeping, thereby indicating that the human is likely not asleep.

One or more temperature sensors, such as temperature sensor 417, may be present within air quality sensor 165. Temperature sensor 417 may be used to measure the ambient temperature in the environment of air quality sensor 165. Measurements made by temperature sensor 417 may be used in conjunction with measurements made by one or more other components of air quality sensor 165, such as air sensor 413, occupancy sensor 414, and sleep sensor 415. For example, the detection by air sensor 413 of the one or more qualities in the air indicating a fire may be corroborated with measurements made by temperature sensor 417 indicating an increase in temperature to determine that a fire is present within the environment. One or more additional temperature sensors that are remote from air quality sensor 165, such as a temperature sensor in smart thermostat 170 and/or a temperature sensor in remote air quality sensor 465, may additionally or alternatively be used to measure the temperature of the ambient environment.

Processing system 419 can include one or more processors. Processing system 419 may include one or more special-purpose or general-purpose processors. Such special-purpose processors may include processors that are specifically designed to perform the functions detailed herein. Such special-purpose processors may be ASICs or FPGAs which are general-purpose components that are physically and electrically configured to perform the functions detailed herein. Such general-purpose processors may execute special-purpose software that is stored using one or more non-transitory processor-readable mediums, such as random access memory (RAM), flash memory, a hard disk drive (HDD), or a solid state drive (SSD) of air quality sensor 165.

Processing system 419 may output information for presentation to electronic display 411. Processing system 419 can receive information from the various sensors, such as air sensor 413, occupancy sensor 414, sleep sensor 415, ambient light sensor 416, and temperature sensor 417. For example, processing system 419 may receive an indication from air sensor 413 that a pollutant was detected within the vicinity of air quality sensor 165. Processing system 419 can perform bidirectional communication with network interface 412, mobile device 140, and/or cloud-based air quality server system 110. For example, after receiving the indication from air sensor 413 that a pollutant was detected, processing system 419 may transmit an alert to mobile device 140. The alert may be a push notification generated by an application running on mobile device 140 and configured to communicate with air quality sensor 165. As another example, processing system 419 may receive information from a sensor, such as air sensor 413, indicating that the presence of a pollutant was detected and transmit the indication to cloud-based air quality server system 110. In some embodiments, processing system 419 executes one or more software applications or services stored on or otherwise accessible by air quality sensor 165. For example, one or more components of air quality sensor 165, such as air sensor 413, occupancy sensor 414, sleep sensor 415, ambient light sensor 416, and temperature sensor 417, may include one or more software applications or software services that may be executed by processing system 419.

Cloud-based air quality server system 110 can maintain a residential user account mapped to air quality sensor 165. Alternatively or in addition, a residential user account may be mapped to a structure and the structure may be further mapped to one or more air quality sensors 165. Air quality sensor 165 may periodically or intermittently communicate with cloud-based air quality server system 110. For example, after detecting the presence of a pollutant, air quality sensor 165 may transmit a message to cloud-based air quality server system 110 including an indication that the pollutant was detected and/or the detected concentration of the pollutant. As another example, air quality sensor 165 may receive instructions from cloud-based air quality server system 110 to change an operational mode of the air quality sensor 165. A person may interact with air quality sensor 165 via a computerized device, such as mobile device 140 and/or personal computer 150. Computerized devices may connect with air quality sensor 165 via network 130. In some embodiments, a computerized device, such as mobile device 140, may be able to monitor the status and measurements of air quality sensor 165 remotely via an application running on the computerized device.

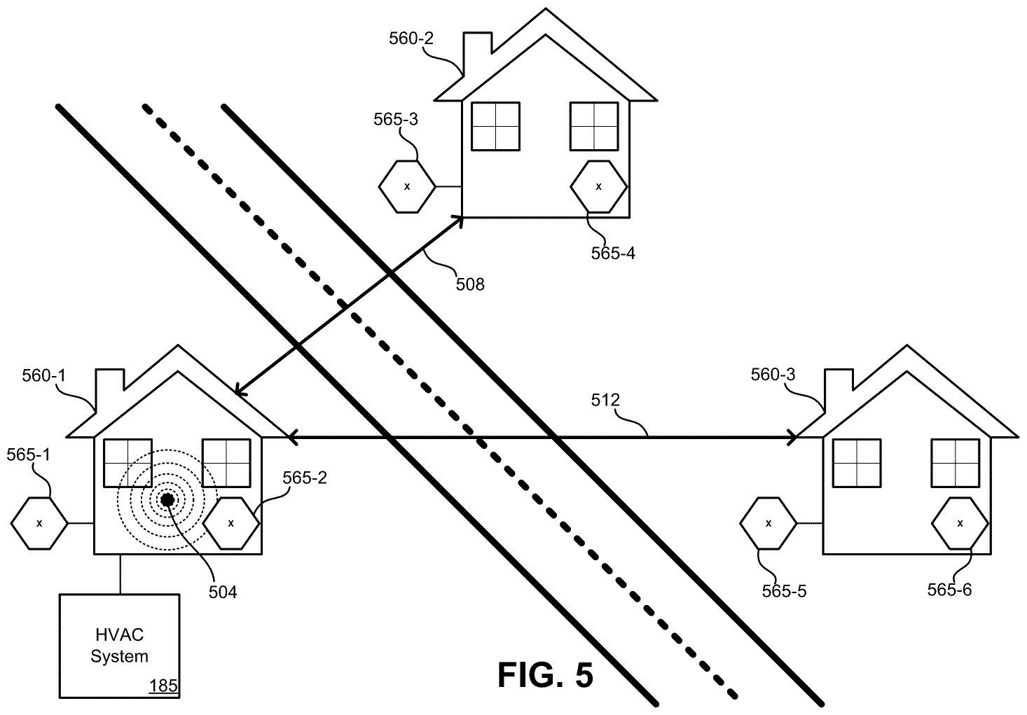

FIG. 5 illustrates an example environment within which a distributed environmental sensor network may be deployed to detect endogenous air pollution within a structure. Endogenous air pollution may be any type of airborne pollution emanating or originating from within a structure. For example, a structure may exhibit endogenous air pollution when there is a gas leak, chemical spill, fire, carbon monoxide buildup, or any similar pollution source within a structure. The detection of endogenous air pollution may be performed by comparing the detected levels of one or more types of pollutant within a structure with the detected levels of the same one or more types of pollutant outside the structure and within a close proximity to the structure. When the detected levels within the structure are higher than the detected levels outside the structure it may be determined that the source of the pollution is inside the structure as opposed to outside structure. This determination may be made more clear by means of illustration as shown in FIG. 5.

As illustrated in FIG. 5, a distributed environmental sensing network may include a plurality of structures 560. Structures 560 may be the same as structures 160 as described further above. For example, structure 560-1 may be a house while structure 560-2 may be a condominium or apartment. Each structure 560 may include one or more air quality sensors 565. Air quality sensors 565 may be the same, or function in a similar manner, as air quality sensor 165 described above. For example, each air quality sensor 565 may be configured to detect the presence, and/or measure a concentration, of one or more types of air pollutants. Each structure 560 may include one or more air quality sensors 565 distributed within the interior and/or around an exterior of structure 560. For example, as illustrated in FIG. 5, structure 560-1 may include air quality sensor 565-2 located within an interior of structure 560-1, while air quality sensor 565-1 is located on or around the exterior of structure 560-1.

Each structure 560 may be associated with a known geographic location. For example, the geographic location may be indicated by a street address, a latitude and longitude, a Military Grid Reference System coordinate, Universal transverse Mercator coordinate, or any similarly suitable location reference. Alternatively, each structure 560 may be mapped to within a certain radius of a known geographic location. For example, each structure 560 may be within a range of 1 mile to 10 miles from a known location. Each structure 560 may be a known distance from another structure 560. For example, as illustrated in FIG. 5, using the known locations of structure 560-1 and structure 560-2, distance 508 between structure 560-1 and structure 560-2 may be determined. Distance 512 between structure 560-1 and structure 560-3 may be determined in a similar fashion. The distances between each structure 560, may be stored in a cloud-based server system, such as cloud-based air quality server system 110 as described above, in feet, meters, yards, miles, or any similarly suitable unit of measurement.

In some embodiments, determining that the source of a pollutant is likely to be within a structure may be based on a comparison of sensor measurements collected by air quality sensors within the structure with air quality sensors within a close proximity to the structure. For example, as illustrated in FIG. 5, air quality sensor 565-2 may detect the presence of a first pollutant within structure 560-1 while air quality sensor 565-1 may not be able to detect the presence of the first pollutant outside structure 560-1. In this case, the likelihood that source 504 of the first pollutant is within structure 560-1 would be higher than the likelihood that source 504 of the first pollutant is outside structure 560-1. Determining that the source of a pollutant is likely to be within a structure may also be based on a difference in the measured concentrations of the pollutant by air quality sensors within the structure and air quality sensors within a close proximity to the structure. For example, air quality sensor 565-2 may measure a higher concentration of a first pollutant within structure 560-1 while air quality sensor 565-1 may measure a lower concentration of the first pollutant outside structure 560-1, thereby increasing the likelihood that source 504 of the first pollutant is within structure 560-1. In some embodiments, comparing sensor measurements collected by air quality sensors in different structures may enhance the accuracy of the determination that the source of a pollutant is likely to be within a structure. For example, if air quality sensor 565-1 is sufficiently close to structure 560-1 it may also detect the presence of the pollutant outside structure 560-1 even though source 504 of the pollutant is within structure 560-1.

In some embodiments, determining that the source of a pollutant is likely to be within a first structure may be based on a comparison of sensor measurements collected by air quality sensors within the first structure and/or within a close proximity to the first structure, and air quality sensors located within a second structure. For example, air quality sensor 565-1 and air quality sensor 565-2 may detect the presence of a first pollutant within structure 560-1 while neither air quality sensor 565-3 nor air quality sensor 565-4 detect the presence of the first pollutant within structure 560-2. By comparing the sensor measurements collected by air quality sensors 565-1 and 565-2 with the sensor measurements collected by air quality sensors 565-3 and 565-4 a determination may be made that source 504 of the first pollutant is within structure 560-1 as opposed to outside structure 560-1.

In some embodiments, the second structure is selected based on the distance between the first structure and the second structure. For example, after detecting a pollutant within structure 560-1, structure 560-2 may be identified for comparison because distance 508 between structure 560-1 and structure 560-2 is less than a predefined distance threshold value. The predefined distance threshold value may be as low as 50 feet or as high as 5 miles or more to enhance the accuracy of the determination. Likewise structure 560-2 may be identified for comparison because distance 508 between structure 560-1 and structure 560-2 is greater than a predefined distance threshold value. In some embodiments, the second structure is identified based on its being between a maximum distance threshold value and a minimum distance threshold value. In some embodiments, the closest structure is selected. For example, structure 560-2 may be selected because distance 508 between structure 560-1 and structure 560-2 is less than distance 512 between structure 560-1 and structure 560-3.

In some embodiments, one or more actions are taken in response to a determination that the source of the pollutant is likely to be within a structure. The one or more actions may include generating and/or issuing a notification for a residential user account associated with the structure. For example, after determining that source 504 of the pollutant is likely to be within structure 560-1, a single-structure alert notification may be issued to an electronic device, such as mobile device 140, associated with the residential user account mapped to structure 560-1. A single-structure alert notification may inform a user of a residential user account that a pollutant was detected and that there is a likelihood that the source of the pollutant is within the structure. The single-structure alert notification may also include suggestions for mitigating the risks posed by the detected pollutant, such as suggesting that occupants vacate the structure and/or open windows and doors to improve circulation within the structure.

Additionally, or alternatively, the one or more actions may include controlling an HVAC system to mitigate the risk posed by the pollutant. For example, a smart thermostat, such as smart thermostat 170, may be caused to control an HVAC system, such as HVAC system 185, to activate an outside air ventilation component. The one or more actions may also include causing one or more air quality sensors distributed within and/or around another structure to change operating modes. For example, after determining that a pollutant is present within structure 560-1, but not within structure 560-2, air quality sensors 565-3 and 565-4 positioned within an/or around structure 560-2 may be caused to change from a normal-sensitivity mode to a high-sensitivity mode, as described above. Changing the operating mode from a normal-sensitivity mode to a high-sensitivity mode may increase the likelihood that the pollutant detected within structure 560-1 will be detected sooner if it spreads to structure 560-2.

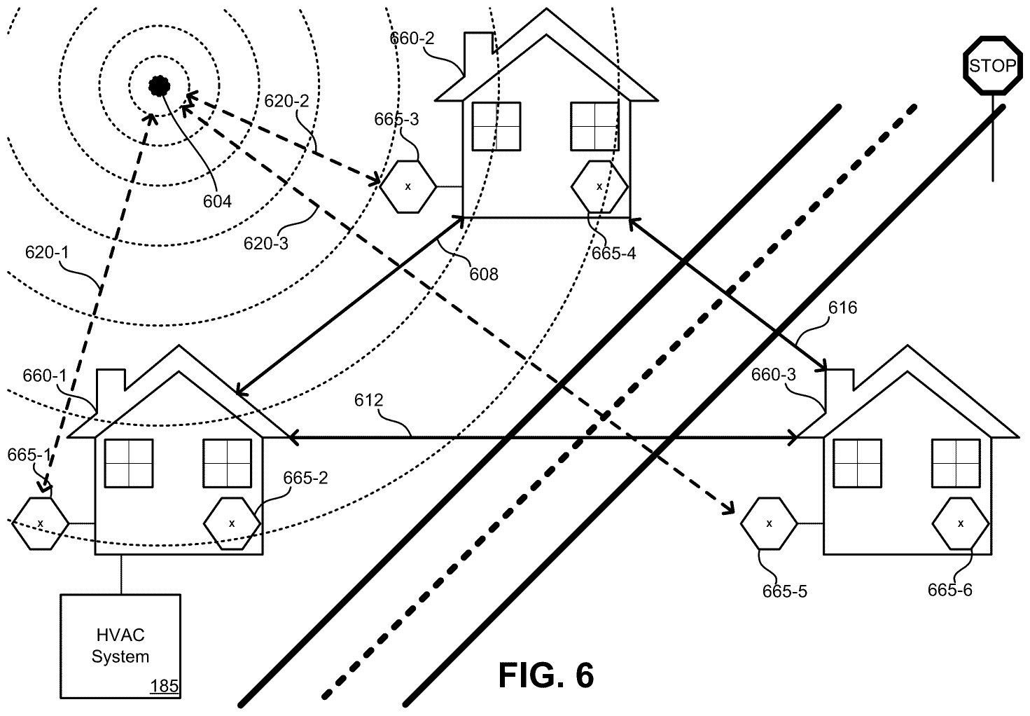

FIG. 6 illustrates another example environment within which a distributed environmental sensor network may be deployed to detect exogenous air pollution within a structure. Exogenous air pollution may be any type of airborne pollution emanating or originating from outside of a structure. For example, exogenous air pollution may emanate from industrial plants, highways or roadways, natural disasters, or any similar pollution source. The detection of exogenous air pollution may be performed by comparing the detected levels of one or more types of pollutant within a structure with the detected levels of the same one or more types of pollutant within another structure or nearby. When a pollutant is detected within multiple structures as opposed to within a single structure, it may be determined that the source of the pollution is external to both structures. This determination may be made more clear by means of illustration as shown in FIG. 6.

As illustrated in FIG. 6, a distributed environmental sensing network may include a plurality of structures 660. Structures 660 may be the same as structures 160 and/or 560 as described further above. For example, structure 660-1 may be a house while structure 660-2 may be a condominium or apartment. Each structure 660 may include one or more air quality sensors 665. Air quality sensors 665 may be the same, or function in a similar manner, as air quality sensors 165 described above. For example, each air quality sensor 665 may be configured to detect the presence, and/or measure a concentration, of one or more types of air pollutants. Each structure 660 may include one or more air quality sensors 665 distributed within the interior and/or around an exterior of structure 660. For example, as illustrated in FIG. 6, structure 660-1 may include air quality sensor 665-2 located within an interior of structure 660-1, while air quality sensor 665-1 is located on or around the exterior of structure 660-1.

Each structure 660 may be associated with a known geographic location. For example, the geographic location may be indicated by a street address, a latitude and longitude, a Military Grid Reference System coordinate, Universal transverse Mercator coordinate, or any similarly suitable location reference. Alternatively, each structure 660 may be mapped to within a certain radius of a known geographic location. For example, each structure 660 may be within a range of 1 mile to 10 miles from a known location. Each structure 560 may be a known distance from another structure 660. For example, as illustrated in FIG. 6, using the known locations of structure 660-1 and structure 660-2 distance 608 between structure 660-1 and structure 660-2 may be determined. Distance 612 between structure 660-1 and structure 660-3 and distance 616 between structure 660-2 and structure 660-3 may be determined in a similar fashion. The distances between each structure 660, may be stored in a cloud-based server system, such as cloud-based air quality server system 110 as described above, in feet, meters, yards, miles, or any similarly suitable unit of measurement.

In some embodiments, determining that the source of a pollutant is likely to be within a structure may be based on a comparison of sensor measurements collected by air quality sensors within the structure with air quality sensors within a close proximity to the structure. For example, as illustrated in FIG. 6, both air quality sensor 665-2 and air quality sensor 665-1 may detect the presence of a first pollutant within an/or around structure 660-1. In this case, the likelihood that source 604 of the first pollutant is outside structure 660-1 would be higher than the likelihood that source 604 of the first pollutant is inside structure 660-1. Determining that the source of a pollutant is likely to be outside a structure may also be based on a difference in the measured concentrations of the pollutant by air quality sensors within the structure and air quality sensors within a close proximity to the structure. For example, air quality sensor 665-1 may measure a higher concentration of a first pollutant outside structure 660-1 while air quality sensor 665-2 may measure a lower concentration of the first pollutant within structure 660-1, thereby increasing the likelihood that source 604 of the first pollutant is outside structure 560-1. In some embodiments, comparing sensor measurements collected by air quality sensors in different structures may enhance the accuracy of the determination that the source of a pollutant is likely to be outside a structure. For example, if air quality sensor 665-1 is sufficiently close to structure 660-1 both air quality sensors 665-1 and air quality sensor 665-2 may detect the presence of the pollutant even though source 604 of the pollutant is within structure 660-1.

In some embodiments, determining that the source of a pollutant is likely to be outside a first structure may be based on a comparison of sensor measurements collected by air quality sensors within the first structure and/or within a close proximity to the first structure, and air quality sensors located within a second structure. For example, air quality sensors 665-1, 665-2, 665-3, 665-4 may each detect the presence of a first pollutant within an/or around structures 660-1 and structure 660-2. By comparing the sensor measurements collected by air quality sensors 665-1 and 665-2 with the sensor measurements collected by air quality sensors 665-3 and 665-4 a determination may be made that source 604 of the first pollutant is outside structure 660-1 as opposed to inside structure 660-1. Similarly, a determination may be made that source 604 of the first pollutant is outside structure 660-2.

In some embodiments, the sensor measurements from the first structure are collected by one or more air quality sensors operating in a normal-sensitivity mode while the sensor measurements from the second structure are collected by one or more sensors operating in a high-sensitivity mode. For example, after detecting the presence of the first pollutant within and/or around structure 660-1 by air quality sensors 665-1 and 665-2 operating in a normal-sensitivity mode, air quality sensors 665-3 and 665-4 may be caused to change operating modes from a normal-sensitivity mode to a high-sensitivity mode. By causing air quality sensors 665-3 and 665-4 to change from the normal sensitivity mode to the high-sensitivity mode, the accuracy and/or speed at which a determination that source 604 of the pollutant is outside structure 660-1 may be increased.

In some embodiments, the second structure is selected based on the distance between the first structure and the second structure. For example, after detecting a pollutant within structure 660-1, structure 660-2 may be identified for comparison because distance 608 between structure 660-1 and structure 560-2 is less than a predefined distance threshold value. The predefined distance threshold value may be 10 miles, 5 miles, 1 mile, or any similarly suitable threshold distance to enhance the accuracy of the determination. Likewise structure 660-2 may be identified for comparison because distance 608 between structure 660-1 and structure 660-2 is greater than a predefined distance threshold value. In some embodiments, the second structure is identified based on its being between a maximum distance threshold value and a minimum distance threshold value. In some embodiments, the closest structure is selected. For example, structure 660-2 may be selected because distance 608 between structure 660-1 and structure 660-2 is less than distance 612 between structure 660-1 and structure 660-3.

In some embodiments, one or more actions are taken in response to a determination that the source of a pollutant is likely to be outside a structure. The one or more actions may include generating and/or issuing a notification for one or more residential user accounts. For example, after determining that source 604 of the pollutant is likely to be outside structure 560-1, a potential-extrinsic-source alert notification may be issued to electronic devices, such as mobile device 140, associated with residential user accounts mapped to structure 660-1 and/or structure 660-2.

Alternatively, or in addition, a potential-extrinsic-source alert notification may be issued to electronic devices associated with residential user accounts mapped to structures within which the pollutant has not been detected. For example, a potential-extrinsic-source alert notification may be issued to electronic devices associated with a residential user account mapped to structure 660-3, allowing preemptive actions to be taken before pollutant breaches structure 660-3. A potential-extrinsic-source alert notification may inform a user of a residential user account that a pollutant was detected within a structure associated with the user and that there is a likelihood that the source of the pollutant is outside the structure. The potential-extrinsic-source alert notification may also include suggestions for mitigating the risks posed by the detected pollutant, such as suggesting that occupants stay within the structure and/or close windows and doors to decrease outside air circulation within the structure.

Additionally, or alternatively, the one or more actions may include controlling an HVAC system to mitigate the risk posed by the pollutant. For example, a smart thermostat, such as smart thermostat 170, may be caused to control an HVAC system, such as HVAC system 185, to deactivate an outside air ventilation component. The one or more actions may also include causing one or more air quality sensors distributed within and/or around another structure to change operating modes. For example, after determining that a pollutant is present within structure 660-1 and structure 660-2, air quality sensors 665-5 and 665-6 positioned within and/or around structure 660-3 may be caused to change from a normal-sensitivity mode to a high-sensitivity mode, as described above. Changing the operating mode from a normal-sensitivity mode to a high-sensitivity mode may increase the likelihood that the pollutant detected within structure 660-1 and structure 660-2 will be detected sooner if it spreads to structure 660-3.

In some embodiments, a location for the source of an exogenous air pollutant may be determined based on the detection of the pollutant within and/or around three or more structures. For example, using distances 608, 612, and 616, and/or the known locations of structures 660, in conjunction with the differences between times 620-1, 620-2, and 620-3 when the presence of the first pollutant was detected within each structure 660, the location of source 604 of the first pollutant may be determined. The location may be determined using a time difference of arrival calculation, or any similarly suitable calculation used in geolocation.

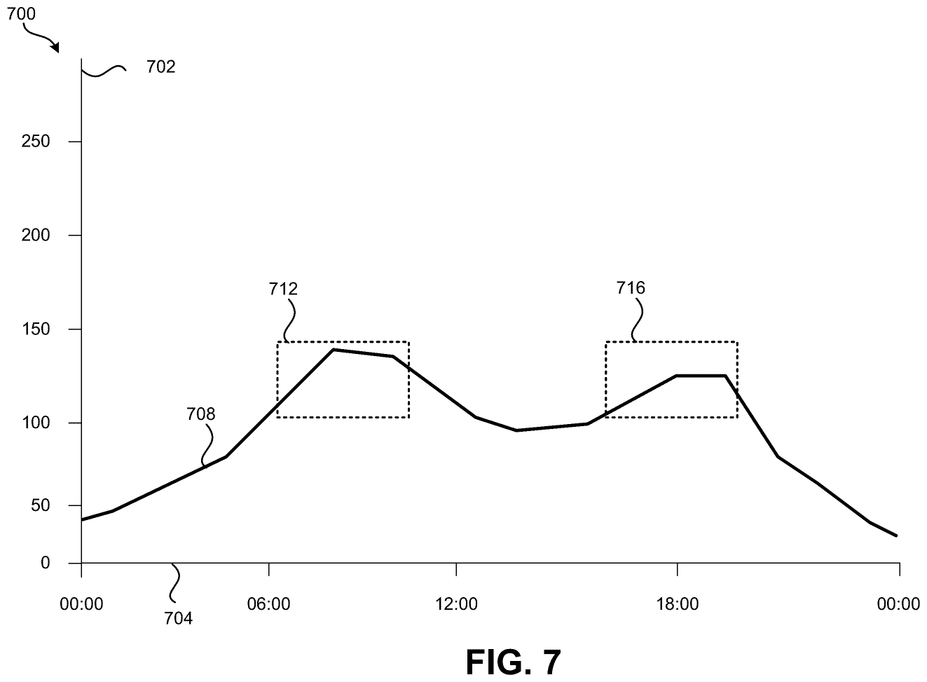

FIG. 7 illustrates a graph 700 of historical air quality. Graph 700 illustrates historical air quality 708 as a function of time. Vertical axis 702 indicates air quality using the air quality index (AQI). However, any similar unit of measurement for air quality may be used, such as parts per million, parts per billion, and/or milligrams by meter cubed. Horizontal axis 704 indicates the time in hours although any unit of time may be used to provide the desired level of granularity. Historical air quality 708 may represent one or more types of pollutant. For example, historical air quality 708 may represent the combined air quality due to a number of measurable pollutants. Alternatively, or in addition, historical air quality 708 may represent a single pollutant and/or type of pollutant.

Historical air quality 708 may represent one or more records of historical air quality over a similar time interval. For example, air quality in a region or specific location may be measured during the same time of day for a plurality of days, and recorded in a database, such as air quality database 317, as described above. Multiple recordings may be made each day for one or more regions and/or locations. Similarly, recordings may include data collected over the course of an entire day and/or during specific times of day. After a sufficient number of recordings have been collected, the recordings may be analyzed to identify trends in the recorded air quality for a region or location. For example, as illustrated in FIG. 7, by analyzing historical air quality 708, peaks 712 and 716 may be identified in a plurality of air quality records. The recordings may be analyzed by a historical data engine, such as historical data engine 314, or a forecast engine, such as forecast engine 316, as described above.

In some embodiments, trends identified in historical air quality recordings are used to determine certain characteristics about the region and/or location where the recordings were collected. For example, peaks 712 and 716 may correspond to an increase in one or more types of pollutant most commonly associated with vehicle exhaust emissions, leading to a determination that the location is likely close to a busy roadway and/or highway. As another example, peaks 712 and 716 may occur at approximately the same time of day each day, leading to a determination that those times correspond with peak rush-hour. In some embodiments, air quality forecasts for a specific location may be predicted based on the identified trends in historical air quality recordings and/or the determined characteristics of the location. For example, based on the identified peaks 712 and 716, an air quality forecast for that location including similar peaks at the same time of day may be generated. In some embodiments, the predicted air quality for a location may be used to generate notifications and/or suggestions for residential user accounts mapped to structures near the location. For example, a notification may be issued to one or more electronic devices, such as mobile device 140, associated with one or more residential user accounts advising users when to keep doors and/or windows closed to correspond with times of increased low air quality.

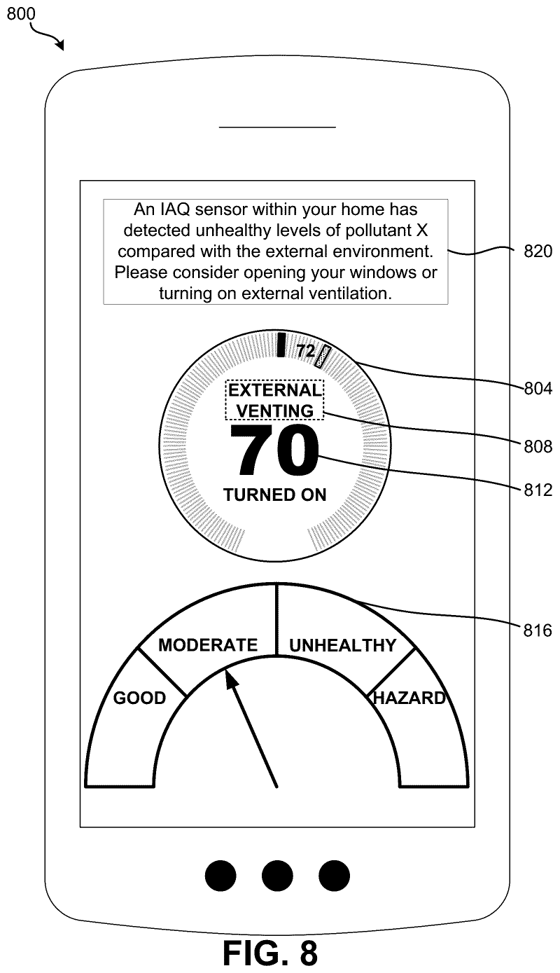

FIG. 8 illustrates an embodiment of an interface 800 for monitoring a distributed environmental sensing network. In some embodiments, interfaces for monitoring the distributed environmental sensing network may be displayed on one or more types of electronic devices, such as mobile device 140 and/or personal computer 150 as described above. Interface 800 may be accessed by executing a software application running on an electronic device and/or by visiting a webpage using a web browser. For example, interface 800 may be a homepage of a software application executed on a mobile device, such as mobile device 140.

Interface 800 may be used to display one or more types of information, such as alerts, notifications, the status of one or more sensors and/or devices, collected sensor measurements, air quality information, and any similarly suitable information. For example, interface 800 may be configured to display banner notification 820 indicating that an alert notification was issued for the residential user account associated with mobile device 140 on which interface 800 is being displayed. In some embodiments, an application running on the electronic device may cause interface 800 to display a pop-up dialog, a badge, an alert, or any other suitable notification method to alert a user that one or more pollutants were detected and the potential source of the one or more pollutants. In response to receiving a selection from a user associated with banner notification 820, interface 800 may display additional information regarding the alert notification, such as the type of pollutant that was detected and/or suggestions for mitigating the risks posed by the detected pollutant.

As another example, interface 800 may display smart thermostat status 804 and air quality status 816. Smart thermostat status 804 may indicate current ambient temperature 812 as measured by a smart thermostat, such as smart thermostat 170 as described above. Smart thermostat status 804 may also indicate current operating mode 808 of the smart thermostat. Air-quality status 816 may indicate the overall air quality in the vicinity of, and as measured by, one or more air quality sensors, such as air quality sensor 165 as described above. Alternatively, or in addition, air quality status 816 may indicate the current measurements of one or more types of pollutant.

Users may access interface 800 by logging in with user credentials associated with a particular residential user account. For example, after opening an application and/or visiting a website, a user may be prompted to enter the user credentials on a login page. After logging in, the information available in interface 800 may be specific to the particular residential user account. For example, each residential user account may be associated with a unique combination of air quality sensors, smart thermostats, and/or other smart devices. Interface 800 may be modified to display information for each unique combination associated with each residential user account.

In some embodiments, one or more aspects of interface 800, are interactive. For example, interacting with smart thermostat status 804 may allow a user to adjust a setpoint temperature associated with the smart thermostat and/or cause an external ventilation component of an HVAC system controlled by the smart thermostat to activate and/or deactivate. As another example, interacting with air quality status 816 may allow a user to adjust the granularity of information displayed in association with the current air quality, change operating modes of one or more air quality sensors, add new air quality sensors, and/or remove existing air quality sensors.

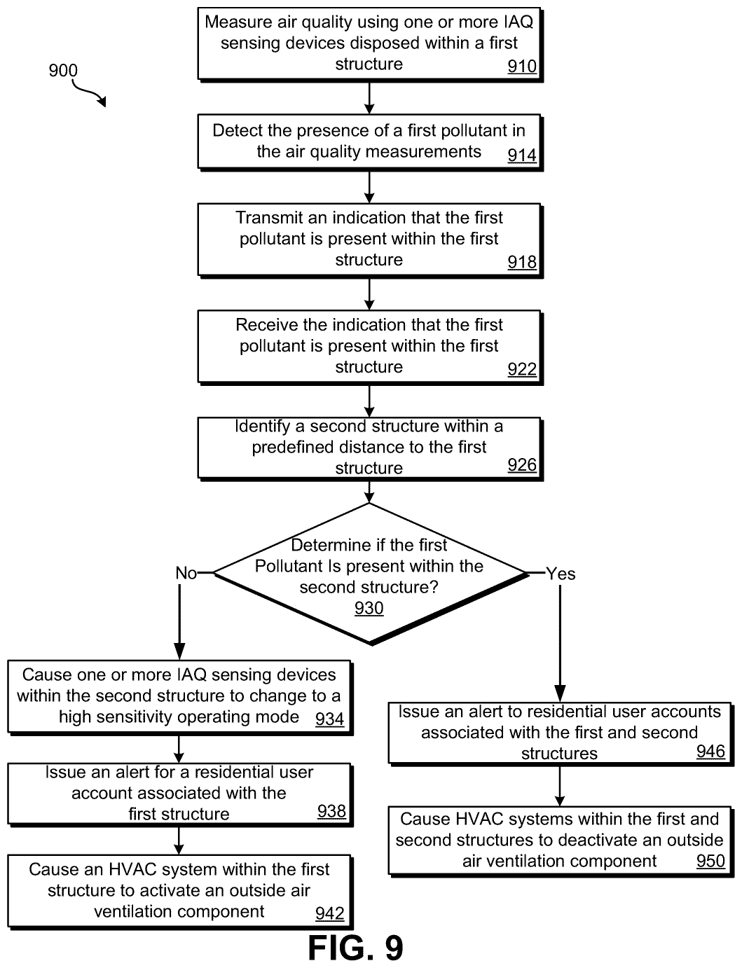

Various methods may be performed using the systems detailed in FIGS. 1-4, supra, to manage distributed environmental sensor networks as detailed in relation to FIGS. 5-8, supra. FIG. 9 illustrates an embodiment of a method 900 for managing a distributed environmental sensor network. In some embodiments, method 900 may be performed by a cloud-based air quality server system, such as cloud-based air quality server system 110 as described in relation to FIG. 3, supra. For example, processing system 318 of cloud-based air quality server system 110 may execute software from one or more modules such as sensor management module 313, historical data engine 314, account management module 315, and/or forecast engine 316. In some embodiments, various steps of method 900 may be performed by one or more air quality sensors, such as air quality sensor 165 as described in relation to FIG. 4, supra. For example, processing system 419 of air quality sensor 165 may execute software from one or more modules such as air sensor 413, occupancy sensor 414, sleep sensor 415, ambient light sensor 416, and/or temperature sensor 417. In some embodiments, some steps of method 900 may be performed by a cloud-based air quality server system, such as cloud-based air quality server system 110 while other steps are performed by air quality sensors, such as air quality sensor 165.

Method 900 may include, at block 910, measuring air quality using one or more indoor air quality (IAQ) sensing devices disposed within a first structure. The one or more IAQ sensing devices may be the same or function in a similar manner as air quality sensor 165 as described above. For example, the one or more IAQ sensing devices may be configured to measure the concentration of one or more pollutants. The concentration of the one or more pollutants may be measured in parts per million, parts per billion, or any similarly suitable unit of measurement for monitoring air quality. The one or more IAQ sensing devices may include one or more operational modes. For example, each IAQ sensing device may include a normal sensitivity mode and/or a high sensitivity mode. In some embodiments, the rate at which measurements are sampled is adjusted based on the current operational mode. For example, the sampling rate may be lower in a normal sensitivity mode as compared with a high sensitivity mode. Alternatively, or in addition, the operational modes may adjust a threshold measurement value at which it is determined that a pollutant is present.

The one or more IAQ sensing devices may be disposed within a structure, such as structure 160, as described above. For example, the first structure may be a house, condominium, apartment, office building, or any similarly suitable structure designed for human occupancy. The one or more IAQ sensing devices may be disposed throughout an interior and/or an exterior of the first structure. For example, IAQ sensing devices may be placed in each room of a house, in addition to multiple locations throughout an exterior of the house. The structure may be associated with a residential user account controlled and/or managed by the cloud-based server system, such as account management module 315 of cloud-based air quality server system 110. Account management module 315 may associate and/or store one or more characteristics of the first structure with the residential user account. For example, the residential user account may include characteristics such as the geographic location of the structure, the size of the structure, the number and/or placement of one or more IAQ sensing devices throughout the structure, and any similarly suitable detail relevant to the detection and/or management of air quality in and around the structure. One or more electronic devices, such as mobile device 140 and/or personal computer 150, may also be associated with a residential user account.

At block 914, the presence of a first pollutant in the air quality measurements is detected. Each measurement may be analyzed to determine whether a pollutant has been detected. The presence of a pollutant may be identified when the measurable concentration and/or amount of the pollutant indicated by a measurement rises above a threshold value and/or when there is any detectable amount of the pollutant (e.g., a threshold value of zero). In some embodiments, each pollutant may have a distinct threshold value corresponding with an acceptable amount of the pollutant within an environment. For example, the threshold value for carbon dioxide may be greater than the threshold value for carbon monoxide due to the increased health risks at lower concentration levels of carbon monoxide as compared with carbon dioxide. In some embodiments, the presence of the first pollutant is detected after a sustained period of increased measurements of the pollutant. For example, a brief increase in the concentration of a pollutant may not indicate the presence of the pollutant, while a prolonged increase in the concentration of a pollutant may indicate the presence of the pollutant.

At block 918, an indication that the first pollutant is present within the first structure is transmitted. For example, one or more of the IAQ sensing devices may transmit an indication that the first pollutant is present within the structure to a cloud-based server system, such as cloud-based air quality server system 110 as described above. The indication may be transmitted via a network, such as network 130 as described above. The indication may include one or more pieces of information, such as an identification of the particular pollutant that was detected, the measured concentration of the pollutant, a unique identifier for the IAQ sensing device, a unique identifier for the first structure, the location of the IAQ sensing device within the structure, and/or the geographic location of the IAQ sensing device. In some embodiments, the indication that the first pollutant is present within the first structure is transmitted apart from existing routine and/or scheduled transmissions. For example, the one or more IAQ sensing devices may transmit a status update at periodic intervals throughout the day. As another example, the one or more IAQ sensing devices may transmit a single status update at the end of each day. Status updates may include some or all of the collected measurements made throughout the day and/or since the last status update was transmitted. In either of the above cases, the indication that the first pollutant is present within the first structure may be transmitted as a separate packet or message.

At block 922, the indication that the first pollutant is present within the first structure is received. For example, a cloud-based server system, such as cloud-based air quality server system 110 as described above, may receive the indication that the first pollutant is present within the first structure from one or more of the IAQ sensing devices. The received indication may be received and/or analyzed by a specialized process and/or module, such as sensor management module 313 of cloud-based air quality server system 110 as described above. In some embodiments, the indication may be analyzed against other air quality data, such as air quality data received from environmental agency data system 120 as described above. For example, after receiving the indication that the first pollutant is present within the first structure, sensor management module 313 may determine, based on available air quality data, that increased levels of the first pollutant are expected within the area around the IAQ sensing devices and a source of the first pollutant is already known.

In some embodiments, after receiving the indication that the first pollutant is present within the first structure, the indication may be stored and/or otherwise associated with the residential user account. For example, account management module 315 may determine that the source IAQ sensing device of the indication is associated with a particular residential user account. As another example, account management module 315 may determine that the source IAQ sensing device is associated with the structure, and the structure is associated with a particular residential user account. After determining the particular residential user account, additional information associated with the residential user account may be used to make further determinations and such or take additional actions. For example, the residential user account may indicate one or more characteristics about the structure within which the IAQ sensing devices are disposed, such as the size and/or location of the structure, and/or one or more electronic devices associated with the residential user account.

At block 926, a second structure within a predefined distance to the first structure is identified. After analyzing the indication that the first pollutant is present within the first structure, a second structure may be identified in order to compare the air quality sensor measurements collected in the second structure with the sensor measurements collected within the first structure. In some embodiments, any structure within a predefined distance to the first structure may be identified as the second structure. The predefined distance may be a maximum distance such as 10 miles, 5 miles, 1 mile, or any similarly suitable maximum distance. In some embodiments, the second structure is identified based on its being between a maximum distance threshold value and a minimum distance threshold value. In some embodiments, the closest structure is selected.

The second structure may be identified from a plurality of structures associated with one or more residential user accounts. For example, account management module 315 may be able to identify a residential user account associated with a structure located within the predefined distance to the first structure based on a location stored in a residential user account associated with the first structure. Multiple structures may be identified as being within a predefined distance to the first structure. In this case, sensor management module 313 and/or account management module 315 may apply additional filtering criteria to select the second structure. For example, the closest structure of the multiple structures may be selected as the second structure. As another example, a structure with more IAQ sensing devices may be selected over a structure with fewer IAQ sensing devices.

After identifying the second structure, a request for air quality data may be transmitted to one or more IAQ sensing devices associated with the second structure. The IAQ sensing devices may be distributed within and/or around the second structure. The request may include a general request for all air quality data collected within a predefined time frame prior to receiving the request.

Alternatively, or in addition, the request may include a specific request for the current measurements of the first pollutant.

At block 930, it is determined whether the first pollutant is present within the second structure. For example, sensor management module 313 may determine whether any of the IAQ sensing devices located within the second structure have transmitted an indication that the first pollutant is present within the second structure over a predetermined length of time. The predetermined length of time may be 5 minutes, 10 minutes, 30 minutes, or any similarly suitable amount of time in the past. If none of the IAQ sensing devices within the second structure have transmitted an indication, then it may be determined that the first pollutant is not within the second structure. In some embodiments, determining whether the first pollutant is present within the second structure includes analyzing recent sensor measurements collected by one or more IAQ sensing devices located within the second structure. For example, sensor management module 313 may analyze the most recent report generated by the one or more IAQ sensing devices located within the second structure. The report may include measurements of one or more detectable pollutants. As another example, sensor management module 313 may analyze one or more previous reports generated by the one or more IAQ sensing devices located within the second structure. The one or more previous reports may include collected measurements of for a predefined time interval. For example, one or more reports may be analyzed until the collected measurements cover the past five minutes, 15 minutes, 30 minutes, one hour, or any similarly suitable period of time.

If the first pollutant is not present within the second structure, method 900 may include, at block 934, causing one or more IAQ sensing devices within the second structure to change to a high-sensitivity operating mode. The one or more IAQ sensing devices may include one or more operational modes. For example, each IAQ sensing device may include a normal sensitivity mode and/or a high sensitivity mode. In some embodiments, the rate at which measurements are sampled is adjusted based on the current operational mode. For example, the sampling rate may be lower in a normal sensitivity mode as compared with a high sensitivity mode. Alternatively, or in addition, the operational modes may adjust a threshold measurement value at which it is determined that a pollutant is present. In some embodiments, causing the one or more IAQ sensing devices to change to a high-sensitivity operating mode includes changing modes for a predetermined length of time. For example, the one or more IAQ sensing devices may change to the high-sensitivity operating mode for the next five minutes, 15 minutes, 30 minutes, one hour, or any similarly suitable amount of time. After the length of time has expired, the one or more IAQ sensing devices may independently change back to a previous operating mode if the first pollutant has not been detected.

In some embodiments, IAQ sensing devices within one or more additional structures are caused to change to a high-sensitivity operating mode. For example, sensor management module 313 and/or account management module 315 may determine that there are one or more structures within a predefined distance to the first structure and cause one or more IAQ sensing devices within each structure to change from a normal sensitivity operating mode to a high-sensitivity operating mode. The various operating modes may correspond to different sampling rates of the IAQ sensing devices. For example, the high-sensitivity mode may cause the IAQ sensing devices to take samples more frequently than the normal sensitivity mode. The predefined distance may be any distance from the first structure, such as 1 mile, 5 miles, 10 miles, or any similarly suitable distance.

At block 938, an alert is issued for a residential user account associated with the first structure. For example, a single-structure alert notification may be issued to an electronic device, such as mobile device 140, associated with the residential user account mapped to the first structure. A single-structure alert notification may inform a user of a residential user account that a pollutant was detected within the structure and that there is a likelihood that the source of the pollutant is within the structure. Determining the likelihood that the source of the pollutant is within the structure may be based, at least in part, on the determination that the pollutant was not present within the second structure. The single-structure alert notification may also include suggestions for mitigating the risks posed by the detected pollutant, such as suggesting that occupants vacate the structure and/or open windows and doors to improve circulation within the structure.

In some embodiments, no identifying information relating to the second structure and/or a residential user account associated with the second structure is made available to users of the first residential user account. For example, the alert issued to the residential user account associated with the first structure may indicate that the first pollutant was detected within the first structure but has not been detected outside of the first structure. In some embodiments, no alerts are issued for the second residential user account after determining that the first pollutant is not present within the second structure. Issuing an alert for the residential user account associated with the first structure may be accomplished without the knowledge and/or involvement of users of the second residential user account. For example, the one or more IAQ sensing devices within the second structure may respond to requests from the cloud-based air quality server system and/or change operating modes without otherwise indicating that they are responding to requests for information and/or changing operating modes.

At block 942, an HVAC system within the first structure is optionally caused to activate an outside air ventilation component. For example, a smart thermostat, such as smart thermostat 170, may be caused to control an HVAC system, such as HVAC system 185, to activate an outside air ventilation component. Activating an outside air ventilation component may help facilitate the replacement of the polluted air within the first structure with fresh air from outside the structure. The outside air ventilation component may include one or more fans and/or vents distributed within the structure such that external air may be drawn into the first structure while exhausting the polluted air out of the structure.

Returning to block 930, if the first pollutant is present within the second structure, method 900 may include, at block 946, issuing an alert for residential user accounts associated with the first and second structures. The alert may be a potential-extrinsic-source alert notification. A potential-extrinsic-source alert notification may inform a user of a residential user account that a pollutant was detected within a structure associated with the user and that there is a likelihood that the source of the pollutant is outside the structure. A potential-extrinsic-source alert notification may also include suggestions for mitigating the risks posed by the detected pollutant, such as suggesting that occupants stay within the structure and/or close windows and doors to decrease outside air circulation within the structure.

In some embodiments, a potential-extrinsic-source alert notification is issued to electronic devices, such as mobile device 140, associated with residential user accounts mapped to the first structure and the second structure. In some embodiments, a potential-extrinsic-source alert notification may be issued to electronic devices associated with residential user accounts mapped to additional structures. The additional structures and/or residential user accounts may be identified based on the distance between a structure mapped to a residential user account and the first and/or second structures. For example, the additional structures may include any structure within a predefined distance, such as within a range of 100 feet to 10 miles away from the first and/or second structures. The additional structures may or may not include one or more IAQ sensing devices. For example, a potential-extrinsic-source alert notification may be issued to electronic devices associated with a residential user account mapped to a third structure within which the first pollutant has not been detected by one or more IAQ sensing devices. As another example, a potential-extrinsic-source alert notification may be issued to electronic devices associated with any residential user account mapped to a structure within a predefined distance of the first and/or second structures, regardless of the presence of IAQ sensing devices within the structure. By issuing alerts for residential user accounts associated with structures within which a pollutant has not been detected preemptive actions may be taken before the pollutant reaches the structure.

At block 950, HVAC systems within the first and second structures are optionally caused to deactivate an outside air ventilation component. For example, smart thermostats, such as smart thermostat 170, within the first and second structures may be caused to control HVAC systems, such as HVAC system 185, to deactivate an outside air ventilation component. Deactivating an outside air ventilation component may help reduce the amount of the pollutant able to enter the first and second structures.

As illustrated by way of example in FIGS. 5-9, distributed environmental sensing system may be managed and monitored in order to detect the presence of one or more pollutants within one or more structures, determine whether the source of the pollutant is likely to be within a structure, and take proactive measures to mitigate risks posed by the detected pollutants. The distributed environmental sensor network may accomplish each step without exposing and/or otherwise sharing personally identifiable information (PII) associated with a residential user account with users of other residential user accounts.

In many cases, pollutants are generated by inorganic processes or sources, such as a power plant, or a gas leak. In some cases, pollutants may come from either an inorganic process or an organic process. For example, carbon dioxide may be produced by burning fossil fuels, or by human respiration. As another example, volatile organic compounds (VOCs) may originate from anthropogenic sources such as evaporated fuels and/or solvents like acetone, or they may originate from one or more bodily emissions, such as respiration and/or dermal excretion. Human production or emission of VOCs may be due to a number of reasons. For example, stress may cause an increased amount of sweat, leading to the additional production of detectable VOCs associated with body odor. As another example, humans may exhale alcohol after drinking alcoholic beverages. In many cases, the production of VOCs by humans is harmless, but in some cases, it may be an indication of an underlying health issue or condition. For example, an increased production of acetone may correlate to Diabetic ketoacidosis.

In some cases, the methods and systems for detecting and measuring VOCs produced by bodily functions may be time consuming and/or use more costly and specialized lab equipment. In some embodiments, one or more sensors in an environmental sensing system, as described above and further herein, are used to detect and measure VOCs produced by bodily functions. For example, by monitoring sensor measurements from one or more sensors over a period of time, the system may be configured to detect an increase in one or more VOCs during that time and further determine that the source of the VOCs is a particular human without the active participation or interaction from the human. After detecting and measuring the one or more VOCs, a report may be generated for the human providing them with specialized information regarding their health and informed suggestions for taking additional actions in response.

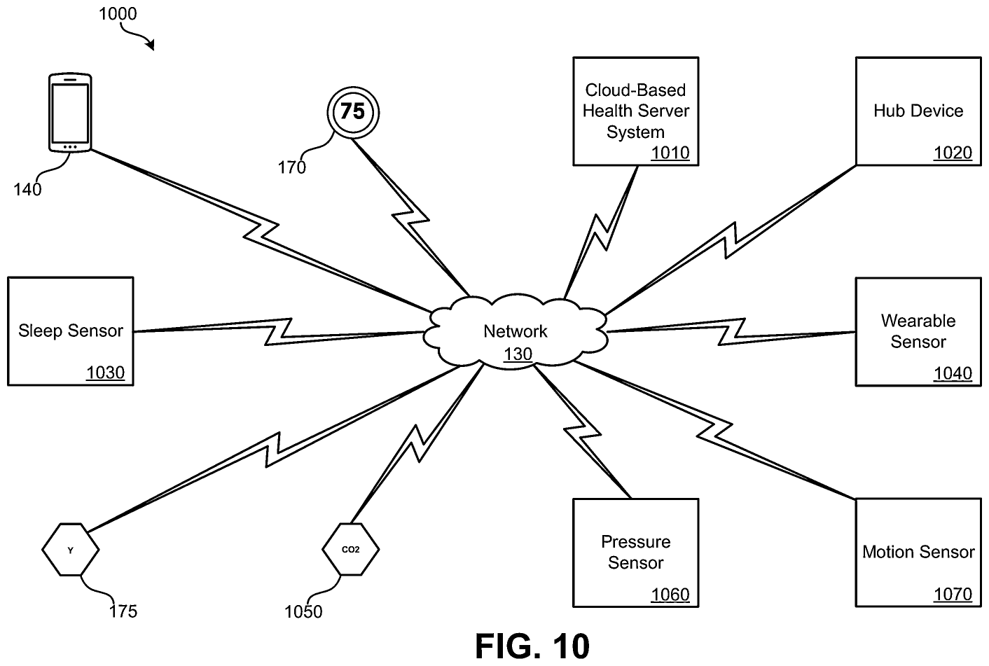

Further detail regarding the detection and measurement of VOCs and the generation of health assessments from an environmental sensor network system is provided in relation to the figures. FIG. 10 illustrates an embodiment of a system 1000 for generating health assessments based on detected volatile organic compounds. System 1000 can include: network 130; mobile device 140; smart thermostat 170; cloud-based health server system 1010; hub device 1020; sleep sensor 1030; wearable sensor 1040; VOC sensor 175; carbon dioxide sensor 1050; pressure sensor 1060; and motion sensor 1070. Network 130, mobile device 140, and smart thermostat 170 may function as detailed in relation to FIGS. 1-4, supra. VOC sensor 175 may function as detailed in relation to FIG. 1, supra. One or more components of system 1000 may be included in one or more electronic devices.

For example, sleep sensor 1030, VOC sensor 175, carbon dioxide sensor 1050, pressure sensor 1060, and/or motion sensor 1070 may be components of an electronic device or sensor system, such as air quality sensor 165 as described in relation to FIGS. 1-4, supra. One or more components of system 1000 may be distributed throughout a structure, such as structure 160 as described above, and/or within an enclosed space.

In some embodiments, one or more components of system 1000 may be in communication with cloud-based air quality server system 110 or any component of system 100 described above. Similarly, It should be understood by one of skill in the art, that any combination of components in system 1000 may be included across one or more devices. Similarly, it should be understood that one or more components represented in system 1000 may include duplicates. For example, system 1000 may include a plurality of VOC sensors 175.

Cloud-based health server system 1010 can include one or more processors configured to perform various functions, such as receive and analyze sensor measurements from the one or more other components of system 1000. Cloud-based health server system 1010 can include one or more physical servers running one or more processes. Cloud-based health server system 1010 can also include one or more processes distributed across a cloud-based server system. In some embodiments, cloud-based health server system 1010 is connected over network 130 to any or all of the other components of system 1000. For instance, cloud-based health server system 1010 may connect to VOC sensor 175 to receive VOC measurements collected by VOC sensor 175 over a period of time. Alternatively, or in addition, cloud-based health server system 1010 may connect to hub device 1020 to request and receive sensor measurements collected from one or more components of system 1000.

Cloud-based health server system 1010 may be configured to attribute detected VOCs to a human based on one or more additional inputs from other sensing devices, such as carbon dioxide sensor 1050, pressure sensor 1060, and/or motion sensor 1070. For example, cloud-based health server system 1010 may identify one or more measurements collected from carbon dioxide sensor 1050 indicating that a human is present in the vicinity of carbon dioxide sensor 1050 for a prolonged period of time. Further, cloud-based health server system 1010 may identify an increase in the concentrations of one or more VOCs detected by VOC sensor 175 in the vicinity of carbon dioxide sensor 1050 during the period of time in which the human was present. Finally, the production of the one or more VOCs may be associated with the human based on the determination that the increase in the concentrations of the one or more VOCs coincided with the period of time in which the human was present in the vicinity of the VOC sensor 175.

Cloud-based health server system 1010 may be configured to manage user accounts. For example, cloud-based health server system 1010 may allow anyone to create a user account in order to participate in the detection and measurement of VOCs and/or receive health assessments based on detected VOCs. In some embodiments, a user account is associated with a residential structure, such as structure 160 as described above. User accounts may be the same as, and/or be managed in the similar way as, the residential user accounts managed by account management module 315, as described above. Users may create multiple profiles under a user account for each occupant of a structure associated with the user account. Users may associate one or more sensing devices, such as

VOC sensor 175 and carbon dioxide sensor 1050, with the user account and/or a particular profile of the user account. For example, VOC sensor 175 may be associated with a particular profile based on VOC sensor 175 being positioned within a bedroom of the occupant associated with the profile.

Cloud-based health server system 1010 may be configured to generate health assessments for user accounts based on one or more detected VOCs being attributed to a human associated with the user account. A health assessment may include a report of the particular VOCs detected and attributed to a human. Additionally, or alternatively, a health assessment may include an indication of a person's overall health, which may include a likelihood or prediction that the person may be suffering from one or more health conditions or illnesses. For example, a health assessment may indicate that there is a high likelihood that a person has a viral or bacterial infection, a particular disease, increased body odor, and/or is otherwise producing an abnormal amount of one or more VOCs compared with the average healthy individual. Predicting the likelihood that a person is suffering from a health condition may include identifying an increased emission of a first VOC by humans as a symptom associated with a health risk, such as a disease or infection.

Cloud-based health server system 1010 may also connect to mobile device 140 to transmit a health assessment for a human associated with mobile device 140. For example, after detecting and attributing VOC production to a human associated with mobile device 140, cloud-based health server system 1010 may send a notification to mobile device 140 with an alert indicating that the VOC was detected and any potential health implications associated with the VOC. Cloud-based health server system 1010 may also connect to smart thermostat 170 to send commands indicating how and/or when to control an HVAC system. For example, cloud-based health server system 1010 may send a command to smart thermostat 170 instructing it to adjust a setpoint temperature based on the detection of one or more VOCs produced by a human indicating that the human is either too cold or too warm.

In some embodiments, cloud-based health server system 1010 may be the same as cloud-based air quality server system 110 and/or an extension of cloud-based air quality server system 110. For example, cloud-based health server system 1010 and cloud-based air quality server system 110 may each include one or more processes distributed across a cloud-based server system. Additionally, or alternatively, one or more components of cloud-based air quality server system 110 may support cloud-based health server system 1010. For example, sensor management module 313 may analyze measurements collected by one or more components of system 1000, such as carbon dioxide sensor 1050, pressure sensor 1060, and motion sensor 1070. As another example, account management module 315 may control and manage one or more user accounts associated with one or more humans.

Hub device 1020 may be a computerized device that can communicate with cloud-based health server system 1010 via network 130. Hub device 1020 may also be configured to communicate, via network 130 and/or directly, with any of sleep sensor 1030, wearable sensor 1040, VOC sensor 175, carbon dioxide sensor 1050, pressure sensor 1060, and motion sensor 1070. For example, hub device 1020 may be configured to send and receive communications via any of a variety of custom or standard wireless protocols (Wi-Fi, ZigBee®, 6LoWPAN, Thread®, Bluetooth®, BLE®, HomeKit Accessory Protocol (HAP)®, Weave®, Matter, etc.) and/or any of a variety of custom or standard wired protocols (CAT6 Ethernet, HomePlug®, etc.). In some embodiments, hub device 1020 can serve as an edge router that translates communications between a mesh network and a wireless network, such as a Wi-Fi network. For example, one or more components, such as VOC sensor 175, carbon dioxide sensor 1050, pressure sensor 1060, and/or motion sensor 1070, may form a mesh network and transmit data to hub device 1020 for relay to cloud-based health server system 1010 for analysis.

In some embodiments, one or more components of system 1000 may be included in hub device 1020. For example, hub device 1020 may include any combination of VOC sensor 175, carbon dioxide sensor 1050, pressure sensor 1060, and/or motion sensor 1070. A user can interact with an application executed on hub device 1020 to control or interact with smart thermostat 170, VOC sensor 175, sleep sensor 1030, wearable sensor 1040, carbon dioxide sensor 1050, pressure sensor 1060, and/or motion sensor 1070. For example, the user of hub device 1020, may monitor the status of smart thermostat 170 or send heating and cooling instructions to smart thermostat 170 that will in turn cause an HVAC system to provide heating or cooling to the user's home. Hub device 1020 may also be connected over network 130 to cloud-based air quality server system 110. For example, cloud-based air quality server system 110 may send notifications to mobile device 140 about the air quality surrounding or inside the user's home or location. Hub device 1020 may also be connected over network 130 to cloud-based health server system 1010. For example, hub device 1020 may transmit collected sensor measurements from one or more sensors to cloud-based health server system 1010 and receive health assessment notifications and/or updates based on an analysis of the collected sensor measurements. Notifications or updates may be in the form of a text message, an email, or a notification through an application. Hub device 1020 may include an electronic display configured to display the notifications and/or updates.

Sleep sensor 1030 may be one or more sensors configured to detect when a person is sleeping and monitor the quality of sleep. For example, sleep sensor 1030 may include one or more of a heart rate monitor, a breathing rate monitor, a brain activity monitor, a motion detection sensor, and eye activity monitor or any other similar sensor capable of monitoring and detecting measurable characteristics of the sleeping human. In some embodiments, measurements from sleep sensor 1030 may be used in connection with collected VOC measurements to generate a health assessment for a human. For example, after detecting the presence of one or more VOCs, sensor data collected by sleep sensor 1030 may be used to determine that a human is in fact the source of the detected VOCs and that the human is asleep. As another example, after detecting the presence of a VOC, a sleep quality assessment may be generated based on sensor data collected by the sleep sensor. The sleep quality assessment may be used in combination with the collected VOC measurements to corroborate an initial health assessment based on the VOC measurements alone.

Wearable sensor 1040 may be one or more sensors configured to detect and/or monitor various vital signs and bodily functions relating to the health of the wearer. For example, wearable sensor 1040 may include one or more of a heart rate monitor, a breathing rate monitor, a pulse oximeter, a brain activity monitor, a motion detection sensor, an eye activity monitor, and/or any similarly suitable sensor for monitoring human activity. In some embodiments, wearable sensor 1040 may include, or be included in, sleep sensor 1030. For example, sleep sensor 1030 may be a component of wearable sensor 1040 configured to monitor and analyze various measurements associated with sleep. Wearable sensor 1040 may be further configured to determine a current activity level for the wearer. The current activity level may indicate whether the wearer is sedentary, performing light activity, and/or performing strenuous activities. The collected measurements of the wearer and/or any correlated determinations, such as an activity level, may be used in connection with collected VOC measurements to generate a health assessment for the wearer. For example, an increase in the detected levels of one or more VOCs, such as body odor, may be disregarded after determining that they increased as a result of the wearer performing strenuous activities. In some embodiments, wearable sensor 1040 is in communication with one or more other components of system 1000. For example, wearable sensor 1040 may be paired over Bluetooth® with mobile device 140. Wearable sensor 1040 may also transmit collected measurements to, and receive health assessment notifications from, cloud-based health server system 1010.

Carbon dioxide sensor 1050 may be an air quality sensor, such as air quality sensor 165 as described above, configured to detect and measure the concentration of carbon dioxide and any other number of pollutants within the vicinity of the sensor. Alternatively, carbon dioxide sensor 1050 may be a standalone sensing device configured to detect and measure the concentration of carbon dioxide alone. Carbon dioxide sensor 1050 may measure the concentration of carbon dioxide in parts per million (PPM) and/or parts per billion (PPB).

Carbon dioxide sensor 1050 may be configured to determine, based on an accumulation of carbon dioxide over a period of time, that a human is present in the vicinity of the carbon dioxide sensor 1050. For example, carbon dioxide sensor 1050 may detect a steady rate of increase in the measured concentrations of carbon dioxide consistent with the presence of at least one human and determine that at least one human is present within the vicinity of the sensor. As another example, carbon dioxide sensor 1050 may detect an increase in the concentration of carbon dioxide from a first steady state concentration to a second steady state concentration consistent with human occupancy. In some embodiments, the rate at which carbon dioxide builds up, and/or the steady state concentration, within the environment consistent with human occupancy are preprogrammed values. Alternatively, or in addition, those values may be determined using a trained machine-learning model by analyzing historical carbon dioxide measurements. A machine learning model may be trained with additional inputs such as collected measurements from one or more other components of system 1000.

Carbon dioxide sensor 1050 may be configured to determine, based on an accumulation of carbon dioxide over a period of time, that the carbon dioxide sensor 1050 is within an enclosed space and/or that the enclosed space is substantially sealed. An enclosed space may be an area that is surrounded on all sides by a physical barrier such as walls, ceilings, and floors. Additionally, or alternatively, an enclosed space may be an area with limited entry and egress. Examples of enclosed spaces may include: cars, recreational vehicles (e.g., a camper), houses, offices, apartments, planes, and/or trains. An enclosed space may be substantially sealed when the concentration of one or more gases within the enclosed space is inhibited and/or unable to reach equilibrium with the concentration of the one or more gases outside the enclosed space. Alternatively, or in addition, an enclosed space may be substantially sealed when the air pressure within the enclosed space is not affected by air pressure changes outside the enclosed space. Determining that an enclosed space is substantially sealed may include determining that the volume of air flowing into and/or out of the enclosed space is below a threshold flow rate. Carbon dioxide may accumulate, and/or the concentration of carbon dioxide may increase, within an enclosed space that is substantially sealed due to the normal respiration of a human occupant. Therefore, determining that an enclosed space is substantially sealed may also include detecting an accumulation and/or increase in the concentration of carbon dioxide within the enclosed space.

In some embodiments, the size of the enclosed space may be used to further determine that the enclosed space is substantially sealed and/or that there is a human present within the enclosed space. For example, carbon dioxide sensor 1050 may be programmed with the size and/or volume of the enclosed space. Alternatively, the volume of the enclosed space may be determined by the dimensions of the enclosed space within which the carbon dioxide sensor 1050 is positioned. The volume may then be stored in a memory of carbon dioxide sensor 1050 and/or in a memory of cloud-based health server system 1010. Based on the volume of the enclosed space, the expected rate at which carbon dioxide will increase within the enclosed space when a human is present may be adjusted up or down. For example, the rate at which carbon dioxide will increase in a small space may be faster than in a larger space.

Pressure sensor 1060 may be an electronic device configured to measure the atmospheric pressure in the vicinity of the electronic device. Pressure sensor 1060 may include one or more barometers. Pressure sensor 1060 may measure atmospheric pressure in bars and/or millimeters/inches of mercury. In some embodiments, the atmospheric pressure measured by pressure sensor 1060 may be used to help determine that an enclosed space is substantially sealed. For example, if the detected air pressure measured by pressure sensor 1060 changes by more than a threshold value, this may correspond to a window or door in the enclosed space being closed, thereby sealing the enclosed space. Alternatively, or in addition, if the detected air pressure measured by pressure sensor 1060 does not change, or changes by less than a threshold value, over the course of a predefined interval of time, this may correspond with no windows or doors being opened in the enclosed space during the predefined interval of time.

Motion sensor 1070 may be an electronic device with one or more sensors configured to detect motion within an environment, such as an enclosed space. For example, motion sensor 1070 may include one or more of a radar sensor, a lidar sensor, a photographic sensor, an infrared sensor, or any similarly suitable sensor capable of detecting motion within an environment. In some embodiments, motion detected by motion sensor 1070 is used to help determine that a human is present within an enclosed space. For example, motion detected by motion sensor 1070 may be combined with measurements collected by carbon dioxide sensor 1050 to determine that a human is in fact present within the enclosed space.

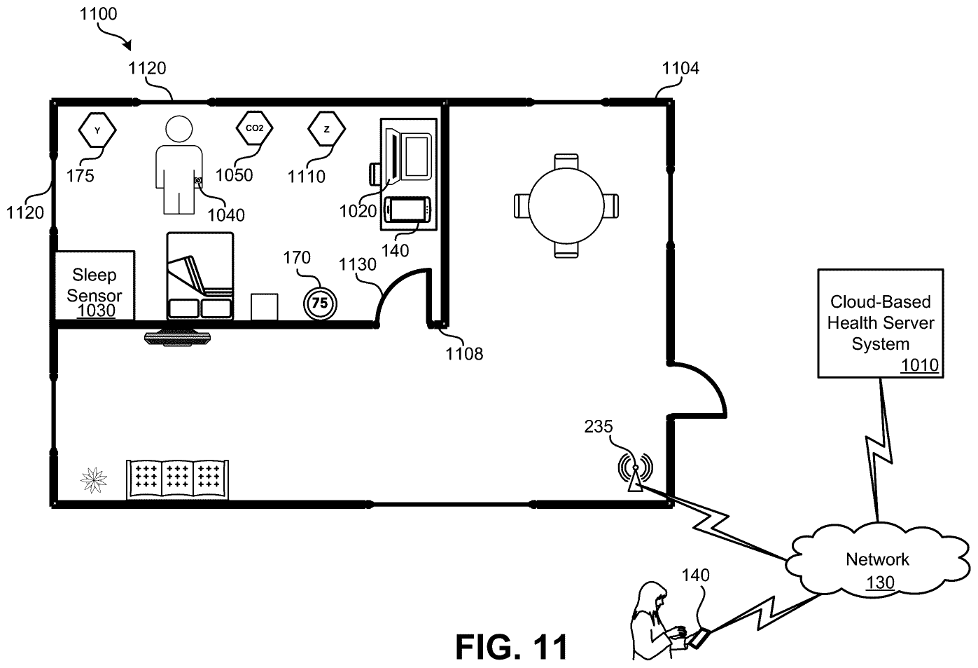

FIG. 11 illustrates an example of an environment 1100 within which one or more of the devices, methods, systems, services, and/or computer program products described further herein can be applicable. The depicted environment 1100 includes structure 1104. Structure 1104 can include, e.g., a house, condominium, apartment, office building, garage, or mobile home, and may be similar to structure 160 as described above. The environment 1100 may include devices, such as VOC sensors 175, smart thermostat 170, hub device 1020, sleep sensor 1030, wearable sensor 1040, carbon dioxide sensor 1050, sensor device 1110, and wireless router 235 inside the actual structure 1104. Sensor device 1110 may include a pressure sensor, such as pressure sensor 1060, and/or a motion sensor, such as motion sensor 1070 as described above.

Structure 1104 may include one or more enclosed spaces 1108 separated at least partly from each other via one or more walls enclosing the structure and enclosed space 1108 from the sides. Structure 1104 may also include ceilings and walls enclosing the structure from above and below. The walls can include windows 1120 and doors 1130. When each of the windows 1120 and door 1130 are closed, enclosed space 1108 may become substantially sealed, as described above. Devices can be mounted on, integrated with and/or supported by walls and or surfaces within enclosed space 1108. For example, smart thermostat 170 may be mounted on an internal wall of enclosed space 1108 while VOC sensor 175 may be positioned on a surface such as a desk or nightstand.

One or more intelligent, network-connected, multi-sensing devices, such as VOC sensor 175 and carbon dioxide sensor 1050, can detect and measure the concentrations of hazardous substances and/or pollutants within enclosed space 1108, such as VOCs and carbon dioxide. One or more sensor devices, such as sensor device 1110, can detect changes in air pressure and/or the movement of an occupant within enclosed space 1108. The data collected by each of the one or more devices may be provided to a central device and/or service, such as cloud-based health server system 1010, or hub device 1020, for analysis.

In addition to containing processing and sensing capabilities, each of the devices, such as smart thermostat 170, hub device 1020, sleep sensor 1030, wearable sensor 1040, carbon dioxide sensor 1050, sensor device 1110, can be capable of data communications and information sharing with each of the other devices, as well as to any cloud server or any other device that is network connected anywhere in the world, such as mobile device 140 as described above. The devices can send and receive communications via any of a variety of custom or standard wireless protocols (Wi-Fi, ZigBee®, 6LoWPAN, Thread®, Bluetooth®, BLE®, HomeKit Accessory Protocol (HAP)®, Weave®, Matter, etc.) and/or any of a variety of custom or standard wired protocols (CAT6 Ethernet, HomePlug®, etc.). Each of the devices may also be capable of receiving voice commands or other voice-based inputs from a user, such as the Google Home® interface.

For example, a first device can communicate with a second device via a wireless router 235. A device can further communicate with remote devices via a connection to a network, such as network 130. Through network 130, the device can communicate with a central server or a cloud-computing system, such as cloud-based health server system 1010 and/or cloud-based air quality server system 110. Further, software updates can be automatically sent from the central server or cloud-computing system to the devices (e.g., when available, when purchased, or at routine intervals).

By virtue of network connectivity, one or more of the devices of FIG. 11 can further allow a user to interact with the device even if the user is not proximate to the device. For example, a user can communicate with a device such as mobile device 140. A webpage or app can be configured to receive communications from the user and control the device based on the communications and/or to present information about the device's operation to the user. For example, the user can view the current concentrations of one or more types of pollutant using a computer. The user can be in the structure during this remote communication or outside the structure.

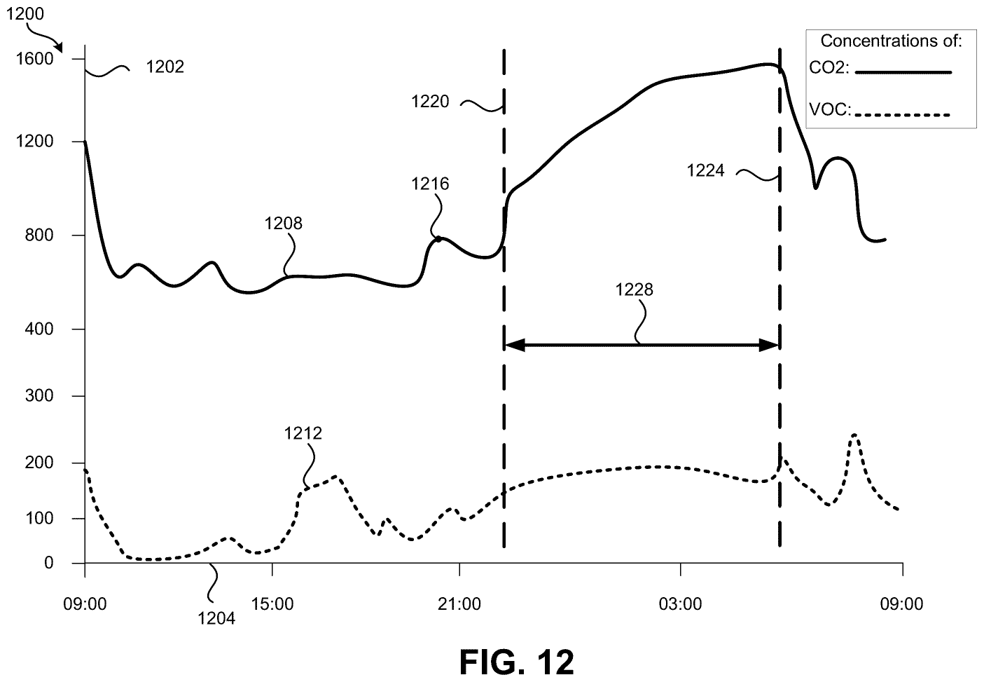

FIG. 12 illustrates a graph 1200 of carbon dioxide and VOC concentrations detected in an enclosed space. Graph 1200 illustrates the measured carbon dioxide concentration 1208 within the enclosed space as a function of time. Graph 1200 also illustrates the measured VOC concentration 1212 within the enclosed space as a function of time. Vertical axis 1202 indicates the concentration in air as parts per million, however, any similar unit of measurement for concentrations of airborne pollutants may be used, such as parts per billion and/or milligrams per meter cubed. Horizontal axis 1204 indicates the time in hours although any unit of time may be used to provide the desired level of granularity. The measured VOC concentration 1212 may represent one or more types of VOCs. For example, VOC concentration 1212 may represent the combined concentrations of all VOCs detectable by the VOC sensor, such as VOC sensor 175. The enclosed space within which the measurements were collected may include a bedroom.

As illustrated in FIG. 12, the measured carbon dioxide concentration 1208 remains at a stable level for the majority of the day and increases sharply between approximately 21:00 and 06:00 the next morning before decreasing again. An initial peak 1216 in carbon dioxide concentration 1208 at approximately 21:00 may coincide with when a human entered the enclosed space and/or an increased amount of activity by a human within the enclosed space corresponding to increased respiration by the human. The steady increase in carbon dioxide concentration 1208 starting at time 1220 may coincide with when the human closed a door of the enclosed space, thereby causing the enclosed space to be substantially sealed and restricting the equalization of carbon dioxide from within the enclosed space to outside the enclosed space, as described above. The following decrease in carbon dioxide concentration 1208 starting at time 1224 may coincide with a time when the human opened the door, thereby causing the enclosed space to no longer be substantially sealed and the carbon dioxide built up within the enclosed space to equalize with the carbon dioxide outside the enclosed space.

As further illustrated in FIG. 12, time interval 1228 indicates the time during which carbon dioxide concentration 1208 steadily increased towards a peak at time 1224. A cloud-based health server system, such as cloud-based health server system 1010 as described above, may be configured to analyze the measured carbon dioxide concentration 1208 and determine that a human was present during time interval 1228. Alternatively, or in addition, one or more other devices, such as mobile device 140 and/or hub device 1020, may be configured to determine that a human was present during time interval 1228. Either a cloud-based health server system or another device may further be configured to determine that the human was asleep during time interval 1228. Determining that the human was present and/or asleep during time interval 1228 may include analyzing additional data collected by one or more other sensors, such as sleep sensor 1030, wearable sensor 1040, pressure sensor 1060, and/or motion sensor 1070, as described above. For example, a detected movement by motion sensor 1070 at time 1220 and an indication from sleep sensor 1030 that the human was asleep during some portion of interval 1228 may be used to determine that the human was present during the entirety of time interval 1228 and asleep for at least a majority of time interval 1228.

FIG. 12 also illustrates a slight increase in VOC concentration 1212 over time interval 1228 in addition to occasional increases in VOC concentration 1212 throughout the collected measurements. A cloud-based health server system, such as cloud-based health server system 1010 as described above, may be configured to analyze the measured carbon dioxide concentration 1208 in conjunction with the measured VOC concentration 1212 and attribute the slight increase in VOC concentration 1212 over time interval 1228 to a human as opposed to some other anthropogenic source. For example, after determining that the human was present and/or asleep within the enclosed space based on the measured carbon dioxide concentration 1208 and inputs from other devices, cloud-based health server system 1010 may attribute the slight increase in VOC concentration 1212 to the human. Alternatively, or in addition, one or more other devices, such as mobile device 140 and/or hub device 1020, may be configured to attribute the increase in VOC concentration 1212 to the human. After attributing the increase in VOC concentration 1212 to the human, additional steps may be taken, such as generating a health assessment for the human based on the particular VOC that was attributed to the human.

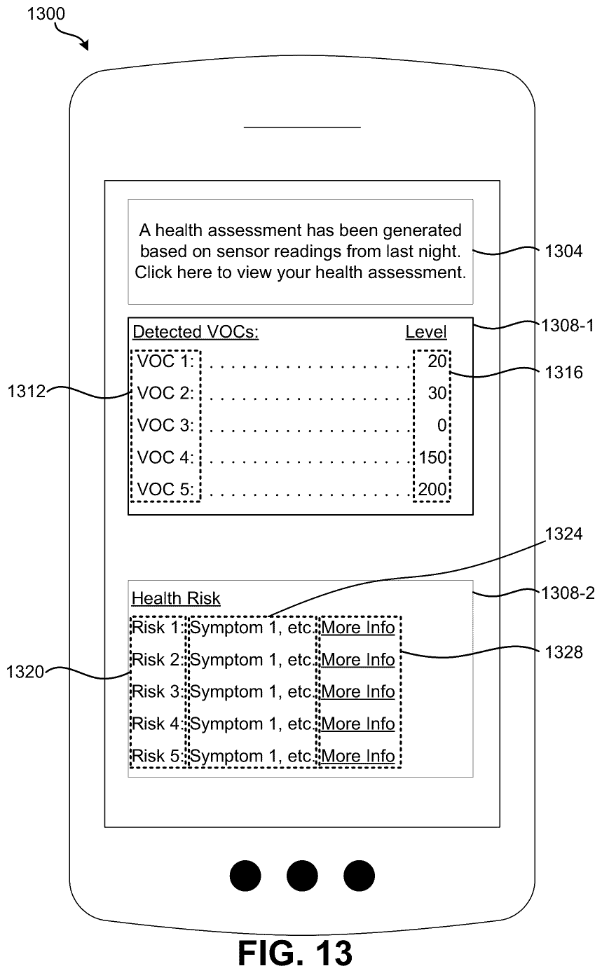

FIG. 13 illustrates an embodiment of an interface 1300 for viewing generated health assessments based on detected volatile organic compounds. In some embodiments, interfaces for viewing generated health assessments based on detected volatile organic compounds may be displayed on one or more types of electronic devices, such as mobile device 140 and/or hub device 1020 as described above. Interface 1300 may be accessed by executing a software application running on an electronic device and/or by visiting a webpage using a web browser. For example, interface 1300 may be a homepage of an application executed by mobile device 140 and/or hub device 1020.

Interface 1300 may be configured to display one or more types of information in various formats relating to the detection of VOCs within an enclosed space and/or generated health assessments based on the detected VOCs. For example, as illustrated in FIG. 13, interface 1300 may be configured to display banner notification 1304 indicating that a health assessment has been generated for the user associated with the electronic device. In some embodiments, an application running on the electronic device may cause interface 1300 to display a pop-up dialog, a badge, an alert, or any other suitable notification method to alert a user that a health assessment has been generated for the user.

As illustrated in FIG. 13, interface 1300 may also be configured to display one or more containers 1308 of related information. For example, interface 1300 may include VOC detection container 1308-1 and/or health assessment container 1308-2. Each container 1308 may include one or more fields for displaying related data. For example, VOC detection container 1308-1 may include detected VOCs 1312 indicating the VOCs that were measured and/or detected by a VOC sensor, and VOC levels 1316 indicating the concentration of each VOC that was measured or detected. VOC levels 1316 may indicate the concentration of VOCs in parts per million, parts per billion, a grade (e.g., low, medium, high), or any similarly suitable measurement for displaying the concentration of measured VOCs.

As another example, health assessment container 1308-2 may include health risks 1320, symptoms 1324, and/or additional links 1328. Health risks 1320 may indicate the overall health risk that was identified based on the detected concentrations of one or more VOCs, such as detected VOCs 1312. The health risks may be any type of health risk such as an underlying disease or illness, and/or an infection, such as a viral or bacterial infection, as described above.

Symptoms 1324 may indicate common symptoms associated with each identified health risk. Symptoms 1324 may also indicate other symptoms being experienced by a user and further associated with each identified health risk. For example, a user may provide the system with one or more symptoms the user is currently experiencing via a separate interface, and the health assessment may be generated and/or updated based on the symptoms provided by the user. The symptoms may also be identified from measurements collected by other sensing devices, such as a sleep sensor or wearable sensor. For example, measurements collected by a sleep sensor may indicate that the user is experiencing below average sleep quality. The indication of below average sleep quality may then be used in conjunction with the detection of one or more VOCs to identify health risks with matching symptoms. As another example, measurements collected by a wearable sensor may indicate that the user has recently experienced higher blood pressure, and heightened heart rate while VOCs associated with body odor have been detected by the VOC sensor. The combination of the increased level of vital signs with the VOCs associated with body odor may be used to identify stress as a health risk associated with all of the detected symptoms.

Additional links 1328 may include links to additional information related to each health risk. Some links may navigate to a new page of the application and/or website. For example, the application may have one or more pages of information for each health risk. Additional links 1328 may also be configured to navigate to an external page and/or website. For example, additional links 1328 may navigate to a dedicated health website or the website of a local physician who specializes in treating that particular health risk.

In some embodiments, VOC detection container 1308-1 and health assessment container 1308-2 are accessible via different pages of an application and/or website. For example, a user may be able to navigate to a sensor status page of an application and/or website in order to view VOC detection container 1308-1 and/or the most recent measurements collected by one or more additional sensing devices, such as carbon dioxide sensor 1050, sleep sensor 1030, wearable sensor 1040, and/or motion sensor 1070. VOC detection container 1308-1 and health assessment container 1308-2 may also be accessible via a single page of an application and/or website. For example, a health assessment page may include both VOC detection container 1308-1 and health assessment container 1308-2. As another example, in response to receiving a selection from a user associated with banner notification 1304, interface 1300 may display the health assessment page.

Users may access interface 1300 by logging in with user credentials associated with a particular user account. For example, after opening an application and/or visiting a website, a user may be prompted to enter the user credentials on a login page. After logging in, the information available in interface 1300 may be specific to the particular user account. For example, each user account may be associated with a unique combination of enclosed spaces and sensing devices. After logging in, interface 1300 may display a home page for a user account including interactive fields for modifying one or more settings and/or features associated with the user account. For example, by navigating to a settings page, a user may be able to add and/or remove sensing devices from the user account, associate existing sensing devices with a different enclosed space of the user account, associate a profile with a different enclosed space of the user account, and/or any other similarly suitable action.

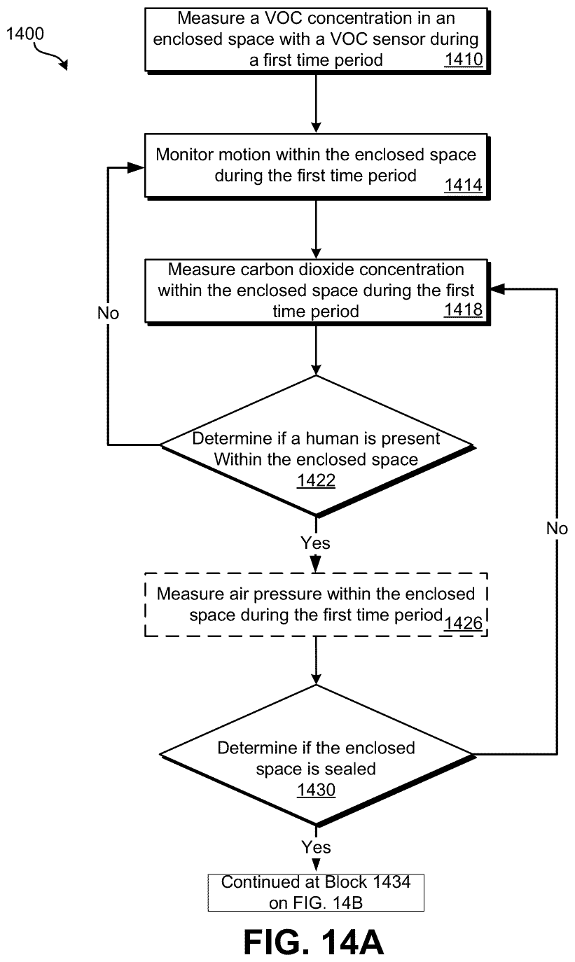

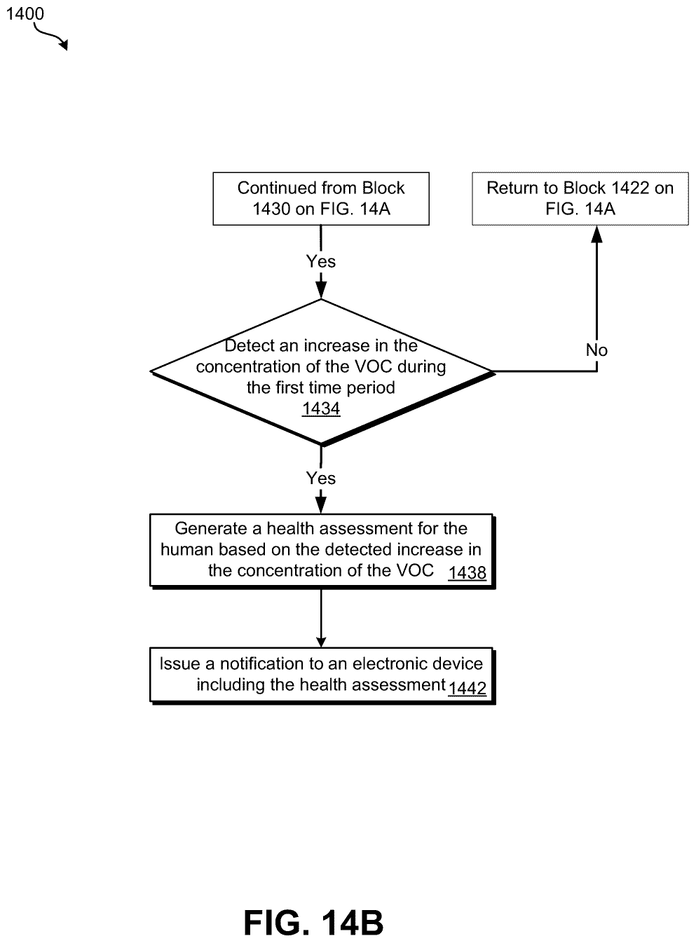

Various methods may be performed using the systems detailed in FIG. 10, supra, to manage the detection and measurement of VOCs and the generation of health assessments from an environmental sensor network system as detailed in relation to FIGS. 11-13, supra. FIGS. 14A and 14B illustrate an embodiment of a method 1400 for generating a health assessment based on detected volatile organic compounds. In some embodiments, method 1400 may be performed by a cloud-based health server system, such as cloud-based health server system 1010 as described in relation to FIG. 10, supra. In some embodiments, various steps of method 1400 may be performed by one or more sensing devices, such as VOC sensor 175, carbon dioxide sensor 1050, pressure sensor 1060, and/or motion sensor 1070 as described in relation to FIG. 10, supra. In some embodiments, some steps of method 1400 may be performed by a cloud-based health server system, such as cloud-based health server system 1010 while other steps are performed by sensing devices, such as VOC sensor 175.

Method 1400 may include, at block 1410, measuring a VOC concentration in an enclosed space with a VOC sensor during a first time period. The VOC sensor may be the same or function in a similar manner as VOC sensor 175 as described above. The VOC sensor may also be configured to measure the concentration of one or more additional VOCs within an enclosed space. The concentration of the one or more VOCs may be measured in parts per million, parts per billion, or any similarly suitable unit of measurement for measuring VOCs. The enclosed space may be disposed within a structure, such as structure 160 as described above. For example, the structure may be a house, and the enclosed space may be a bedroom within the house. The structure may have one or more additional VOC sensors disposed throughout the structure in other enclosed spaces. For example, each bedroom within a structure may have a VOC sensor disposed therein. The structure, the enclosed space, and/or the VOC sensor may be associated with a user account controlled and/or managed by a cloud-based health server system, such as cloud-based health server system 1010 as described above. The enclosed space may be associated with a particular human occupant and/or user profile under the user account. For example, the user account may store a description of the enclosed space in association with a user profile who typically occupies the enclosed space.

At block 1414, motion is monitored within the enclosed space during the first time period. Motion within the enclosed space may be monitored by one or more motion sensors, such as motion sensor 1070 as described above. The motion sensor may be an electronic device with one or more sensors configured to detect motion within the enclosed space. For example, the motion sensor may include one or more of a radar sensor, a lidar sensor, a photographic sensor (e.g., a camera), an infrared sensor, or any similarly suitable sensor capable of detecting motion within an environment.

At block 1418, carbon dioxide concentration within the enclosed space is measured during the first time period. The carbon dioxide concentration may be measured using a carbon dioxide sensor, such as carbon dioxide sensor 1050 as described above. The Carbon dioxide sensor may be an air quality sensor, such as air quality sensor 165 as described above, configured to detect and measure the concentration of carbon dioxide and any other number of pollutants within the vicinity of the sensor. Alternatively, the carbon dioxide sensor may be a standalone sensing device configured to detect and measure the concentration of carbon dioxide alone. The carbon dioxide sensor may measure the concentration of carbon dioxide in parts per million (PPM) and/or parts per billion (PPB).

At block 1422, it is determined whether a human is present within the enclosed space. Determining whether a human is present within the enclosed space may include detecting an accumulation of carbon dioxide over the period of time. For example, a carbon dioxide sensor, such as carbon dioxide sensor 1050, may be configured to determine, based on an accumulation of carbon dioxide over a period of time, that a human is present in the vicinity of the carbon dioxide sensor, and therefore within the enclosed space. Additionally, or alternatively, another device, such as a hub device or a cloud-based health server system, may be configured to analyze the carbon dioxide measurements from a carbon dioxide sensor and detect the accumulation of carbon dioxide over the period of time. In some embodiments, detected movement within the enclosed space is used to help determine that a human is present within an enclosed space. For example, motion detected by motion sensor 1070 may be combined with measurements collected by carbon dioxide sensor 1050 to determine that a human is in fact present within the enclosed space.

The measured carbon dioxide concentrations may indicate a steady rate of increase in the measured concentrations of carbon dioxide consistent with the presence of at least one human and therefore a determination that at least one human is present within the vicinity of the sensor. As another example, the measured carbon dioxide concentrations may indicate an increase in the concentration of carbon dioxide from a first steady state concentration to a second steady state concentration consistent with human occupancy. In some embodiments, the rate at which carbon dioxide builds up, and/or the steady state concentration, within the environment consistent with human occupancy are preprogrammed values. Alternatively, or in addition, those values may be determined using a trained machine-learning model by analyzing historical carbon dioxide measurements for the enclosed space. A machine learning model may be trained with additional inputs such as collected measurements from one or more other components of system 1000. If there has not been any detected motion, and/or an accumulation of carbon dioxide, within the enclosed space, method 1400 may return to either block 1414 or block 1418 to continue measuring the carbon dioxide concentration and/or detect motion within the enclosed space until a determination that a human is within the enclosed space may be made.

If it is determined that a human is present within the enclosed space, method 1400 may optionally include, at block 1426, measuring air pressure within the enclosed space during the first time period. Air pressure within the enclosed space may be measured using a pressure sensor, such as pressure sensor 1060 as described above. The pressure sensor may be an electronic device configured to measure the atmospheric pressure in the vicinity of the electronic device. The pressure sensor may include one or more barometers. The air pressure within the enclosed space may be measured in bars and/or millimeters/inches of mercury. In some embodiments, the atmospheric pressure measured by a pressure sensor may be used to help determine that an enclosed space is substantially sealed. For example, if the detected air pressure measured by pressure sensor 1060 changes by more than a threshold value, this may correspond to a window or door in the enclosed space being closed, thereby sealing the enclosed space. Alternatively, or in addition, if the detected air pressure measured by pressure sensor 1060 does not change, or changes by less than a threshold value, over the course of a predefined interval of time, this may correspond with no windows or doors being opened in the enclosed space during the predefined interval of time.

At block 1430, it is determined whether the enclosed space is substantially sealed. An enclosed space may be an area that is surrounded on all sides by a physical barrier such as walls, ceilings, and floors. Additionally, or alternatively, an enclosed space may be an area with limited entry and egress. Examples of enclosed spaces may include: cars, recreational vehicles (e.g., a camper), houses, offices, apartments, planes, and/or trains. An enclosed space may be substantially sealed when the concentration of one or more gases within the enclosed space is inhibited and/or unable to reach equilibrium with the concentration of the one or more gases outside the enclosed space. Alternatively, or in addition, an enclosed space may be substantially sealed when the air pressure within the enclosed space is not affected by air pressure changes outside the enclosed space. Determining that an enclosed space is substantially sealed may include determining that the volume of air flowing into and/or out of the enclosed space is below a threshold flow rate.

Determining whether the enclosed space is substantially sealed may also include detecting an accumulation of carbon dioxide over the period of time with a carbon dioxide sensor. A carbon dioxide sensor may be configured to determine, based on an accumulation of carbon dioxide over a period of time, that the carbon dioxide sensor is within an enclosed space and/or that the enclosed space is substantially sealed. Carbon dioxide may accumulate, and/or the concentration of carbon dioxide may increase, within an enclosed space that is substantially sealed due to the normal respiration of a human occupant. Therefore, determining that an enclosed space is substantially sealed may also include detecting an accumulation and/or increase in the concentration of carbon dioxide within the enclosed space.

In some embodiments, the size of the enclosed space may be used to further determine that the enclosed space is substantially sealed and/or that there is a human present within the enclosed space. The size and/or volume of the enclosed space may be determined by the dimensions of the enclosed space. The volume may then be stored in a memory of the carbon dioxide sensor and/or in a memory of a cloud-based health server system. Based on the volume of the enclosed space, the expected rate at which carbon dioxide will increase within the substantially sealed enclosed space when a human is present may be adjusted up or down. For example, the rate at which carbon dioxide will increase in a small space may be faster than in a larger space.

Determining whether the enclosed space is substantially sealed may also include monitoring the air pressure within the enclosed space. For example, if the detected air pressure measured by a pressure sensor changes by more than a threshold value, this may correspond to a window or door in the enclosed space being closed, thereby sealing the enclosed space. Alternatively, or in addition, if the detected air pressure measured by a pressure sensor does not change, or changes by less than a threshold value, over the first time period, this may correspond with no windows or doors being opened in the enclosed space during the first time period. If it is determined that the enclosed space is not substantially sealed, method 1400 may return to either block 1426 to continue measuring the air pressure within the enclosed space until a determination that the enclosed space is substantially sealed is made. Alternatively, method 1400 may return to block 1418, for example, if it is determined that a human is no longer present within the enclosed space.

Continuing on FIG. 14B, if it is determined that the enclosed space is substantially sealed, method 1400 may include, at block 1434, detecting an increase in the concentration of the VOC during the time period. Detecting an increase in the concentration of the VOC during the time period may be performed by a cloud-based health server system, such as cloud-based health server system 1010 as described above. For example, cloud-based health server system 1010 may receive one or more measurements collected by a VOC sensor during the first time period and analyze the one or more measurements to determine if there was an increased concentration of the VOC within the enclosed space during the first time period. Additionally, or alternatively, a hub device or other electronic device, such as hub device 1020 and/or mobile device 140, may be configured to analyze measurements collected by one or more VOC sensors and detect an increase in the VOC during the first time period. As another example, the one or more VOC sensors may be configured to detect increases in the concentrations of one or more VOCs. The VOC sensors may include threshold concentration values for one or more VOCs and may create and/or transmit markers associated with times when the concentration of the one or more VOCs rise above the threshold concentration value for each particular VOC.

In some embodiments, detecting and/or analyzing the measurements collected by a VOC sensor is initiated upon determining that a human is within the enclosed space and that the space is substantially sealed. For example, measurements collected by one or more VOC sensors may be stored in a buffer covering a predefined period of time in the past. As new measurements are collected, the oldest measurements may be removed to optimize storage space. After determining that a human is within the enclosed space and that the enclosed space is substantially sealed, an alert or process may be triggered causing the buffered VOC measurements to be analyzed. The alert or process may not be triggered until a predefined amount of time has passed since determining that the human is within the space and that the space is substantially sealed. The predefined amount of time may be based on the amount of time it takes for VOCs to accumulate within a substantially sealed enclosed space. For example, based on the volume of the enclosed space and average respiration rates, VOCs emitted by a human may not accumulate to a detectable level for 2 hours, 4 hours, 6 hours, or more hours since the enclosed space became substantially sealed.

After determining that a human is within the enclosed space, the enclosed space is substantially sealed, and an increase in the concentration of the VOC is detected, the increased concentration may be attributed to the human within the enclosed space. Attributing the detected VOCs to the human may be based on one or more additional inputs from other sensing devices, such as carbon dioxide sensor 1050, pressure sensor 1060, and/or motion sensor 1070 as described above. In some embodiments, the increased concentration of the VOC may be attributed to a human associated with a user account controlled and managed by a cloud-based health server system, such as cloud-based health server system 1010 as described above. For example, a cloud-based health server system may determine that the collected measurements were received from a VOC sensor disposed within a room associated with a profile of a user account and attribute the increased concentration of the VOC to the profile and/or the human associated with the profile.

In some embodiments, if an increase in the VOC was not detected, method 1400 may return to 1422 to again determine if a human is present within the enclosed space. For example, if it is determined that the human has left the enclosed space, the process may start over until a human is again detected within the enclosed space. Alternatively, method 1400 may return to block 1430 to again determine if the enclosed space is substantially sealed. For example, if the human within the enclosed space opens a window or door, the process may start over until it is determined that the human is no longer within the enclosed space, or that the enclosed space is once again substantially sealed.

If an increase in the concentration of the VOC is detected, method 1400 may include, at block 1438, generating a health assessment for the human based on the detected increase in the concentration of the VOC. A health assessment may include a report of the particular VOCs detected and attributed to a human. Additionally, or alternatively, a health assessment may include an indication of a person's overall health, which may include a likelihood or prediction that the person may be suffering from one or more health conditions or illnesses. For example, a health assessment may indicate that there is a high likelihood that a person has a viral or bacterial infection, a particular disease, increased body odor, and/or is otherwise producing an abnormal amount of one or more VOCs compared with the average healthy individual. Predicting the likelihood that a person is suffering from a health condition may include identifying an increased emission of a first VOC by humans as a symptom associated with a health risk, such as a disease or infection.

Generating a health assessment may also include analyzing one or more identifying features associated with the human. Identifying features may include: age, weight, overall fitness, disclosed illnesses or preexisting conditions, recent vital sign measurements such as resting heart rate, resting respiratory rate, blood pressure, and any similarly suitable identifiable features that may aid in diagnosing a health condition. The identifying features may be stored and/or associated with a user account managed by a cloud-based health server system, such as cloud-based health server system 1010 as described above. For example, a user account may be associated with a structure and one or more humans who typically occupy the structure. Additionally, or alternatively, each human may be associated with an individual profile of the user account (e.g., each family member may have a profile associated with a family's user account). Further, identifying features may be provided by a user associated with the user account for each human occupant and/or profile. Additionally, or alternatively, the identifying features may be updated on a periodic basis as new measurements become available, such as new measurements collected by a sleep sensor and/or a wearable sensor. Generating the health assessment may be performed by a central cloud-based server system, such as cloud-based health server system 1010, or another electronic device, such as hub device 1020 and/or mobile device 140.

At block 1442, a notification including the health assessment is issued to an electronic device. The electronic device may be any electronic device, such as mobile device 140 and/or hub device 1020. The electronic device may be associated with the human and/or a profile of a user account. For example, each profile may be associated with a unique mobile device 140. As another example, each profile may be associated with a single shared hub device 1020. After generating the health assessment for a human associated with a profile and/or user account, a notification may be transmitted to the electronic device associated with the human and/or profile. The notification may include a banner notification indicating that a health assessment has been generated prompting a user to navigate to the health assessment. The health assessment may then be displayed on an interface, such as interface 1300 as described above. The interface may be displayed by a software application executed by the electronic device. Additionally, or alternatively, the interface may be accessible as a website or webpage via an internet browser.

It should be noted that the methods, systems, and devices discussed above are intended

merely to be examples. It must be stressed that various embodiments may omit, substitute, or add various procedures or components as appropriate. For instance, it should be appreciated that, in alternative embodiments, the methods may be performed in an order different from that described, and that various steps may be added, omitted, or combined. Also, features described with respect to certain embodiments may be combined in various other embodiments. Different aspects and elements of the embodiments may be combined in a similar manner. Also, it should be emphasized that technology evolves and, thus, many of the elements are examples and should not be interpreted to limit the scope of the invention.

Specific details are given in the description to provide a thorough understanding of the embodiments. However, it will be understood by one of ordinary skill in the art that the embodiments may be practiced without these specific details. For example, well-known, processes, structures, and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing embodiments of the invention. Various changes may be made in the function and arrangement of elements without departing from the spirit and scope of the invention.

Also, it is noted that the embodiments may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered.

What is claimed is:

1. A method for creating health assessments from Volatile Organic Compound (VOC) detection, the method comprising:
measuring, with a VOC sensor, a concentration of a first VOC within an enclosed space during a first time period;
detecting an accumulation of carbon dioxide within the enclosed space during the first time period;
determining, based on the accumulation of carbon dioxide, that a human is present within the enclosed space;
determining, based on the accumulation of carbon dioxide, that the enclosed space is substantially sealed, wherein when the enclosed space is substantially sealed, airflow into and out of the enclosed space is below a threshold value;
detecting, by the VOC sensor, that the concentration of the first VOC within the enclosed space increased during the first time period;
generating a health assessment for the human based on the detected increase in the concentration of the first VOC; and
issuing a notification to an electronic device, the notification including the health assessment.

2. The method for creating health assessments from VOC detection of claim 1, further comprising determining, based on the determination that the enclosed space is substantially sealed and the determination that the human is present within the enclosed space, that the concentration of the first VOC increased due at least in part to one or more bodily emissions by the human including exhaling, sweating, or both.

3. The method for creating health assessments from VOC detection of claim 1, further comprising determining, using a sleep sensor, that the human is asleep during the first time period

4. The method for creating health assessments from VOC detection of claim 3 further comprising:
generating, based on sensor data collected by the sleep sensor, a sleep quality assessment for the human during the first time period; and
wherein generating the health assessment is further based on a combination of the detected increase in the concentration of the first VOC and the sleep quality assessment.

5. The method for creating health assessments from VOC detection of claim 1, wherein generating the health assessment based on the detected increase in the concentration of the first VOC comprises:
identifying an increased emission of the first VOC by humans as a symptom associated with a health risk; and
including an identification of the health risk in the health assessment.

6. The method for creating health assessments from VOC detection of claim 1, wherein measuring the concentration of the first VOC occurs in response to detecting the accumulation of carbon dioxide within the enclosed space.

7. The method for creating health assessments from VOC detection of claim 1, wherein determining that the human is present within the enclosed space is further based on sensing a movement by the human using a motion sensor.

8. The method for creating health assessments from VOC detection of claim 1, wherein determining that the human is present within the enclosed space further comprises detecting a breathing rate, heart rate, or both associated with the human.

9. The method for creating health assessments from VOC detection of claim 1, wherein the method further comprises:
measuring, using an air pressure sensor, a change in air pressure within the enclosed space during the first time period; and
wherein determining that the enclosed space is substantially sealed further comprises determining that the change in air pressure is less than a threshold value.

10. The method for creating health assessments from VOC detection of claim 1, further comprising measuring, with the VOC sensor, concentrations of a plurality of VOCs, wherein the first VOC is included in the plurality of VOCs.

11. A system for creating health assessments from Volatile Organic Compound (VOC) detection, the system comprising:
a VOC sensor configured to collect VOC concentration measurements of a first VOC within an enclosed space;
a cloud-based health server system, comprising:
one or more processors; and
a memory communicatively coupled with and readable by the one or more processors and having stored therein processor-readable instructions which, when executed by the one or more processors, cause the one or more processors to:
receive the VOC concentration measurements collected by the VOC sensor during a first time period;
determine, based on an accumulation of carbon dioxide within the enclosed space during the first time period, that a human is present within the enclosed space;
determine, based on the accumulation of carbon dioxide, that the enclosed space is substantially sealed, wherein when the enclosed space is substantially sealed, airflow into and out of the enclosed space is below a threshold value;
detect, from the VOC measurements, that the concentration of the first VOC within the enclosed space increased during the first time period;
generate, based on the detected increase in the concentration of the first VOC, a health assessment for the human; and
issue a notification to an electronic device, the notification including the health assessment.

12. The system for creating health assessments from VOC detection of claim 11, further comprising a carbon dioxide sensor configured to measure a carbon dioxide concentration within the enclosed space and transmit an indication of the accumulation of carbon dioxide to the cloud-based health server system.

13. The system for creating health assessments from VOC detection of claim 11, further comprising a sleep sensor configured to determine that the human is asleep during the first time period.

14. The system for creating health assessments from VOC detection of claim 11, further comprising a motion sensor configured to sense a movement by the human within the enclosed space.

15. The system for creating health assessments from VOC detection of claim 11, further comprising an air pressure sensor configured to measure a change in air pressure within the enclosed space during the first time period.

16. The system for creating health assessments from VOC detection of claim 11, further comprising a wearable sensor configured to detect a breathing rate, heart rate, or both associated with the human.

17. The system for creating health assessments from VOC detection of claim 11, further comprising a hub device configured to:
3 receive the VOC concentration measurements from the VOC sensor and transmit the VOC concentration measurements to the cloud-based health server system; and
receive carbon dioxide measurements from a carbon dioxide sensor during the first time period and transmit an indication of the accumulation of carbon dioxide to the cloud-based health server system.

18. A non-transitory processor-readable medium, comprising processor-readable instructions configured to cause one or more processors to:
measure a concentration of a first Volatile Organic Compound (VOC) within an enclosed space during a first time period;
detect an accumulation of carbon dioxide within the enclosed space during the first time period;
determine, based on the accumulation of carbon dioxide, that a human is present within the enclosed space;
9 determine, based on the accumulation of carbon dioxide, that the enclosed space is substantially sealed, wherein when the enclosed space is substantially sealed, airflow into and out of the enclosed space is below a threshold value;
detect that the concentration of the first VOC within the enclosed space increased during the first time period;
generate a health assessment for the human based on the detected increase in the concentration of the first VOC; and
issue a notification to an electronic device, the notification including the health assessment.

19. The non-transitory processor-readable medium of claim 18, wherein the processor-readable instructions are further configured to cause the one or more processors to:
determine, based on the determination that the enclosed space is substantially sealed and the determination that the human is present within the enclosed space, that the concentration of the first VOC increased due at least in part to one or more bodily emissions by the human including exhaling, sweating, or both.

20. The non-transitory processor-readable medium of claim 18, wherein the processor-readable instructions to generate the health assessment are further configured to cause the one or more processors to:
identify an increased emission of the first VOC by humans as a symptom associated with a health risk; and
include an identification of the health risk in the health assessment
